(19) United States
(12) Patent Application Publication
(10) Pub. No.: US 2022/0305488 A1
(43) Pub. Date: Sep. 29, 2022

(54) Dynamic Profiling of Antitumor Activity of CAR T Cells Using Micropatterned tumor Array Devices

(71) Applicant: The General Hospital Corporation, Boston, MA (US)

(72) Inventors: Daniel Irimia, Charlestown, MA (US); Xiao Wang, Malden, MA (US); Irene Scarfo, Cambridge, MA (US)

(21) Appl. No.: 17/638,543

(22) PCT Filed: Aug. 28, 2020
(86) PCT No.: PCT/US2020/048494

§ 371 Date: Feb. 25, 2022

Related U.S. Application Data
(60) Provisional application No. 62/892,604, filed on Aug. 28, 2019.

(51) Int. Cl. B01L 3/00; G01N 15/14; G01N 33/50; G01N 33/68

(52) U.S. Cl. CPC B01L 3/50853; G01N 15/1463; G01N 33/505; G01N 33/6845; B01L 2200/0647; B01L 3/50855; G01N 2015/1486; G01N 2015/1488; G01N 2015/1493

(57) ABSTRACT

Provided herein are cell assay devices, methods of assaying the activity of immune cells on target cells, and methods of selecting a treatment for a subject having cancer. Described herein are cell assay devices comprising a biocompatible substrate having an upper surface supporting a plurality of arrays of spots comprising an adhesion-promoting material; a biocompatible membrane having top and bottom surfaces and positioned adjacent to the upper surface of the substrate and defining a plurality of chambers within the membrane between the top surface and the bottom surface of the membrane, wherein the membrane comprises at least two openings in the top surface of the membrane into each chamber to provide access to the chambers.

### CLAIM OF PRIORITY

This application claims the benefit of U.S. Provisional Application Ser. No. 62/892,604, filed on Aug. 28, 2019. The entire contents of the foregoing are incorporated herein by reference.

### FEDERALLY SPONSORED RESEARCH OR DEVELOPMENT

This invention was made with Government support under Grant Nos. EB002503 and GM092804 awarded by the National Institutes of Health. The Government has certain rights in the invention.

### TECHNICAL FIELD

This disclosure relates to cell assay devices, methods for assaying the activity of immune cells on target cells, and methods of selecting a treatment for a subject having cancer.

### BACKGROUND

Cancer immunotherapy based on the engineering of chimeric antigen receptors (CAR) on T cells has emerged as one of the most promising new therapies for patients with B-cell malignancies. Preclinical assessments of essential CAR-T cell functions such as trafficking and cytotoxicity are critical for accelerating the development of highly effective therapeutic candidates. However, current tools for evaluating CAR-T functions lack sufficient precision.

### SUMMARY

Described herein are cell assay devices comprising a biocompatible substrate having an upper surface supporting a plurality of arrays of spots comprising an adhesion-promoting material; a biocompatible membrane having top and bottom surfaces and positioned adjacent to the upper surface of the substrate and defining a plurality of chambers within the membrane between the top surface and the bottom surface of the membrane, wherein the membrane comprises at least two openings in the top surface of the membrane into each chamber to provide access to the chambers; and wherein each chamber in the membrane is aligned with a respective one of the arrays of spots on the substrate; and a frame positioned on the top surface of the membrane, sandwiching the membrane between the frame and the substrate, wherein the frame defines a series of open-ended compartments, one compartment for each of the chambers within the membrane, wherein the compartments are in fluid communication with the chambers via the openings in the membrane.

In some embodiments, the substrate comprises glass, plastic, or silicon.

In some embodiments, the spots are circular, oval, or toroidal in shape.

In some embodiments, the adhesion-promoting material comprises one or more of poly-L-lysine polyacrylamide, a cationic polymer, and a cell adhesion molecule.

In some embodiments, the substrate is substantially planar.

In some embodiments, the plurality of chambers are formed into the bottom surface of the membrane.

In some embodiments, the membrane comprises a polymer. In some embodiments, the polymer comprises one or more of a polydimethylsiloxane, a plastic, or a hydrogel.

In some embodiments, the membrane is permeable. In some embodiments, the membrane is impermeable. In some embodiments, the membrane is semi-permeable.

In some embodiments, the membrane is permeable for molecules and particles having a diameter of from about 1 nm to about 100 μm.

In some embodiments, the membrane has a thickness of about 1.5 mm.

In some embodiments, the spots are between about 150 and about 200 μm in diameter.

In some embodiments, the total number of spots is between about 1 and about 3,000.

In some embodiments, the distance between spots on an array is at least about 35 μm.

In some embodiments, the number of spots on each array is between about 4 and about 100.

In some embodiments, the height of the chamber is between about 50 to about 150 μm.

In some embodiments, the width and depth of the chamber are about 6.4 mm.

In some embodiments, the compartments are about 9.6 mm square.

Also provided herein are methods of assaying the activity of immune cells on target cells. The methods include introducing the target cells into the chamber of a device of the disclosure through one of the at least two membrane opening; permitting the introduced tumor cells to settle onto the adhesive dots and adhere thereto; flushing the device to remove non-adherent tumor cells; introducing an extracellular matrix protein into the chamber of the device; filling the chamber of the device with media; introducing the immune cells into the chamber of the device; and imaging the chamber.

In some embodiments, the target cells are tumor cells. In some embodiments, the tumor cells are cancer tumor cells. In some embodiments, the cancer tumor cells is selected from the group consisting of fibrosarcoma, myxosarcoma, liposarcoma, chondrosarcoma, osteosarcoma, chordoma, malignant fibrous histiocytoma, hemangiosarcoma, angiosarcoma, lymphangiosarcoma, mesothelioma, leukemia, plasmocytoma, multiple myeloma, Hodgkin lymphoma, Non-Hodgkin lymphoma, leiomyosarcoma, rhabdomyosarcoma, squamous cell carcinoma, epidermoid carcinoma, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma, glioblastoma, neuroblastoma, medulloblastoma, malignant meningioma, malignant schwannoma, neurofibrosarcoma, parathyroid carcinoma, medullary carcinoma of thyroid, bronchial carcinoid, oat cell carcinoma, malignant pheochromocytoma, islet cell carcinoma, malignant carcinoid, malignant paraganglioma, melanoma, Merkel cell neoplasm, cytosarcoma phylloides, Wilms tumor, seminoma, dysgerminoma, endodermal sinus tumor, teratocarcinoma, Sertoli-Leydig cell tumor, granulose-theca cell tumor, hilar cell tumor, lipid cell tumor, and combinations thereof.

In some embodiments, the target cells are cancer cells. In some embodiments, the cancer cells are selected from the group consisting of acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, Wilms tumor, or a combination thereof.

In some embodiments, between about 2.5 million and about 7.5 million tumor cells are introduced into the chamber.

In some embodiments, the extracellular matrix protein comprises fibronectin, laminin, and/or collagen.

In some embodiments, the immune cells are selected from T cells, monocytes, macrophages, natural killer cells, neutrophils, or combinations thereof. In some embodiments, the immune cells are T cells. In some embodiments, the T cells are chimeric antigen receptor T cells (CAR T cells).

In some embodiments, the target cells comprise a reporter gene. In some embodiments, the reporter gene encodes a fluorescent protein.

In some embodiments, the immune cells comprise a reporter gene. In some embodiments, the reporter gene encodes a fluorescent protein.

In some embodiments, about 10 to about 3 million immune cells are introduced into the chamber of the device.

In some embodiments, the imaging is time-lapse microscopy.

Provided herein are methods of selecting a treatment for a subject having cancer comprising (a) identifying a subject having cancer; (b) generating a plurality of CAR T cells from T cells harvested from the subject; (c) assaying the activity of a subset of the plurality of CAR T cells by any one of the methods of the disclosure; and (d) selecting a treatment based on the results of said assaying.

In some embodiments, the tumor cells are harvested from the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F depicts micropatterned tumor arrays (MiTA) for the quantification of CAR T cell killing. FIG. 1A is a schematic illustration showing the assembly of the 16 well device, zoom-in of one well with 64 spots, tumor cell patterning in the well, and subsequent CAR T cell loading and imaging. FIG. 1B shows fluorescent microscopic images of a poly-L-lysine spot array printed with the automated liquid dispenser and a zoom-in picture of one spot. The scale bars in the top and bottom panels are 500 μm and 100 μm respectively. FIG. 1C shows a heat-scattered plot of the diameter vs. roundness of spots. The top and side histograms correspond to the roundness and the diameter respectively. The dashed lines indicate the 95% range. N=1310 islands, N=5 experimental repeats. FIG. 1D shows a fluorescent microscopic image showing an array of tumor-cell islands after patterning. The bottom panel is a zoom-in picture of the region indicated with a white dashed square. The scale bars in the left and right panels are 500 μm and 100 μm respectively. N=1080 islands, N=5 repeats. FIG. 1E shows a heat-scattered plot of tumor cell number vs. cell island area. The top and side histograms correspond to cell number and cell island area, respectively. The dashed lines indicate the 95% range. FIG. 1F shows time-lapse fluorescent microscopic images show anti-BCMA CAR-T cells and BCMA tumor cell islands. The CAR T cells start initially uniformly distributed and end concentrated on top of the tumor cell islands after 14 h. The scale bar is 200 μm.

FIG. 2 is a photograph showing the 64 well plate platform for high-throughput, multiplexed screening. The 64 well plate contains 4 slides (i). Each slide has 16 wells (ii). Each well contains 64 tumor islands (iii).

FIGS. 3A-3H depict endpoint evaluation of overall CAR-T antitumor efficacy using MiTA. FIG. 3A shows schematics of second generation anti-BCMA chimeric antigen receptor construct. FIG. 3B shows expanded T cells from healthy donors included variable anti-BCMA CAR expression with mean transduction of 44%. (N=3 donors, bars represent SEM). FIG. 3C shows FACS plot of RPMI 8226 multiple myeloma cell line stained with anti-BCMA antibody or isotype control. FIG. 3D shows BCMA-anti-BCMA-CAR interactions mediate tumor cells recognition and killing by CAR T cells. FIG. 3E shows fluorescent microscopic images showing the snapshots of the interaction of CAR T cells (red) and tumor cell islands (green) at 0 (i) and 18 h (ii). In control experiments, UTD T cells had limited interaction with tumor cells after 18 h. The scale bars are 200 μm. FIG. 3F shows heat-density scatter plots of tumor cell island area vs. tumor cell number for tumor cells alone at 0 h and 18 h, tumor cells with UTD T cells at 18 h, and tumors with CART cells at 18 h. The number of experiments (N) and the number of tumor cell islands (N) are indicated on each graph. For example, N=3 and N=97 indicate 3 repeats and a total number of 97 islands. FIG. 3G shows the average tumor cell area and average cell number for tumor cells, in the presence and absence of UTD or CART cells (****p<0.0001, one-way ANOVA analysis). FIG. 3H shows percentage of surviving tumor cells at 18 h alone, with UTD T cells, and with four CAR T cells derived from 4 different healthy donors (****p<0.0001, one-way ANOVA analysis, N indicates the number of tumor cell islands measured). N indicates the number of islands.

FIGS. 4A-4H depict dynamic profiling of the CART antitumor activity using MiTA. FIG. 4A shows fluorescent microscopic time-lapse images show the morphological changes of tumor cell islands without T cells (control), with (UTD) T cells, or with CAR-T cells, from 0-16 h. The scale bar is 200 μm. FIG. 4B shows an array of heatmaps showing the progress of tumor-cell elimination measured by percentage of surviving tumor cells and the remaining area of the tumor over 18 h, with CAR-T cells, with UTD T cells, and without T cells. Each heatmap consists of data from 24 individual spots. FIG. 4C shows average percentage of survival tumor cells and FIG. 4D shows average percentage of tumor area over time at various E:T (N=24 islands per condition). FIG. 4E shows killing rate over time at various E:T calculated from (c) and (d). The solid line and dashed line represent the moving average (subsize=2) of the killing rate calculated by the % survival tumor cells and % remaining tumor area correspondingly. The dark and light arrows on the x-axis indicate the starting point of elimination of individual tumor cells and shrinkage of tumor spots correspondingly. The black arrows on the y-axis indicate the peak killing rates. FIG. 4F shows percentage of survival tumor cells, and FIG. 4G shows percentage of remaining tumor area over time with CAR T cells transduced from 3 different donors (N=24 islands per donor). FIG. 4H shows killing rate calculated from (FIG. 4F, solid line) and (FIG. 4G, dashed line) over time for different donors.

FIGS. 5A-5L depict CAR T cell trafficking and clustering promote tumor-cell killing. FIGS. 5C-FG and FH-FL display readouts grouped by different E:T ratios (FIGS. 5C-5G) or different donors (FIGS. 5H-5L). FIG. 5A shows time-lapse fluorescent microscopic images demonstrate the trafficking of CAR-T cells, the formation of CAR-T cluster, and the shrinkage of the CAR-T cluster on top of the tumor cells over time. The scale bar is 200 μm. FIG. 5B shows time-lapse images highlighting the gradual clustering of CAR T cells and wrapping of tumor cells. The scale bar is 200 μm. FIG. 5C shows the area of CAR T cells on the spot over time (left panel) and the calculated trafficking rate (right panel) at E:T=2.5, 5 and 10 (N=24 islands per condition). The red arrows indicate the plateau of trafficking. The black arrows indicate the time when the trafficking rate decreases to 0. FIG. 5D shows fold change of CAR T cell area at 18 h over 0 h at the 3 E:Ts. FIG. 5E shows the area of CAR T clusters (left) and the corresponding number (right) of clusters on the spots (area>1000 μm²) over time at the 3 E:Ts (N=24 islands per condition). FIG. 5F shows the ratio of CAR T cluster area to total area over time (N=24 islands per condition). FIG. 5G shows a graph showing the correlation between the trafficking of CART cells and shrinking of tumor cells during 18 h at the 3 E:Ts. 0, 2, 4, 8 and 18 h time points are highlighted with colored dots. The black arrows indicate the starting time of tumor cell killing. The density of dots along the X and Y directions reflects the rate of CAR T trafficking and the rate of tumor shrinkage correspondingly (N=24). FIG. 5H shows the area of CAR T cells on the spot over time (left panel) and the calculated trafficking rate (right panel) for three donors (N=24 islands per donor). The arrows indicate the time when the trafficking rate decreases to 0 for donor 1, 3 and 2. FIG. 5I shows fold change of CART cell area at 18 h over 0 h for the 3 donors. FIG. 5J shows the area of CAR T clusters (left) and the corresponding number (right) of clusters over time for the 3 donors. FIG. 5K shows the ratio of CAR T cluster area to total area over time (N=24 islands per donor). FIG. 5L shows the correlation between the trafficking of CAR T cells and shrinking of tumor cells for the 3 donors.

FIG. 6 shows the ratio of the total CAR T area (left) and the cluster area (right) to the tumor area over time at E:T ratio of 2.5, 5 and 10 (N=24).

FIGS. 7A-7L depict comparing the antitumor activity of two CAR T-cell constructs using MiTA. FIG. 7A shows time-lapse fluorescent images demonstrating the killing of BCMA+/−MM.1s by APRIL and anti-BCMA CART cells during 18 h. The scale bar is 200 μm. CAR T cells and tumor cells are pseudo-colored red and green correspondingly. FIG. 7B show zoom-in images showing (i) the efficient elimination of tumor cells and clustering of CAR T cells and (ii) inefficient killing and scattered CAR T cells at 18 h. FIG. 7C shows quantification of surviving tumor cells and remaining tumor area at 18 h for the 4 combinations of CAR constructs and tumor-cell types (N=24 islands per condition). Each dot represents one island. Percentage of survival tumor cells (FIG. 7D) and remaining tumor area (FIG. 7E) over time. FIG. 7F shows trafficking of CAR T cells to the tumor island over time (N=24 islands per condition). FIG. 7G shows microscopic images (post thresholding) showing clusters of CAR T cells (area>1000 μm²) on a tumor spot at 18 h. The scale bar is 100 μm. FIG. 7H shows the average area of the largest cluster over 24 spots at 18 h. FIG. 7I shows the probability of spots with 1-4 CAR T clusters for the 4 conditions. FIG. 7J shows the correlation between the trafficking of CART cells and the shrinkage of tumor area during 18 h for the 4 conditions. The line highlights the distinct profile of anti-BCMA CAR vs. BCMA-tumor cells (B vs. -). (FIG. 7K) The CAR T cluster area vs. the percentage of remaining tumor area at 18 h. (FIG. 7L) The ratio of CAR cluster to the tumor island areas at 18 h (****p<0.0001, one-way ANOVA analysis).

### DETAILED DESCRIPTION

Here, we describe a micropatterned tumor array (MiTA) that enables detailed and dynamic characterization of CAR-T cell trafficking towards tumor-cell islands and subsequent killing of tumor cells. We show that CAR-T cells often merge into large clusters that envelop and kill the tumor cells with high efficiency. We also measure significant differences between CAR T cells from different donors and between various CAR-T cell constructs. Overall, the assay allows for multi-faceted, dynamic, high-content evaluation of CAR T trafficking, clustering, and killing and could eventually become a useful tool for immune-oncology research and pre-clinical assessments of cell-based immunotherapies.

Chimeric antigen receptors (CARs) are engineered receptors used to reprogram patient's T cells to specifically target tumor cells. Cancer immunotherapy based on CAR T cells has emerged as one of the most promising new therapies for the treatment of patients with B-cell malignancies^{[1-8]}. The antitumor activity of CAR T cells relies on efficient CAR T cell trafficking to cancer niches, recognition of tumor antigen, and potent cytotoxicity towards tumor cells. These biological processes are dynamic and involve collective interactions of CAR T and tumor cells. Comprehensive preclinical assessments of these key processes are of pivotal importance for ensuring CAR T therapeutic efficacy.

Several in vitro assays can measure CAR T cell cytotoxicity towards tumor cells. Chromium (Cr51) release assay is the gold standard for quantifying cytotoxicity in the study of tumor cytolysis^{[9]}. However, this assay only provides simple end-point readouts and does not distinguish the CAR T-mediated killing of target cells from other causes of target cell death. Also, the assay is cumbersome to implement and poses safety challenges because it involves the use of radioactive materials. Assays based on the quantification of cytosolic enzymes such as lactose dehydrogenase (LDH)^{[10]} or glyceraldehyde phosphate dehydrogenase (GAPDH)^{[11]} circumvent the need for radioactive materials. However, these assays fail to distinguish the death of target cells from effector cells, since both release cellular enzymes upon lysis. This problem is avoided when firefly luciferase (Fluc)-expressing cells are employed as targets of effector T cells and the release of Fluc into the medium is a specific measure of target cell lysis. However, despite being widely used, these biochemical assays are limited to quantifying only the bulk responses at a single time point which can hardly elucidate the complex and dynamic antitumor activity of CAR T cells.

The integration of real-time monitoring techniques such as time-lapse imaging^{[12]} and electrical impedance sensors^{[13]} with cell culture plates have enabled the dynamic characterization of the cytolysis process. However, the random arrangement of cells in traditional cell culture dishes prohibits the study of CAR T cell trafficking. Lab-on-a-chip technology such as microfluidic cell culture and organ-on-chips hold great promise for advancing the therapeutic screening of cancer immunotherapies^{[14-18]}. However, they are labor-intensive and difficult to use^{[19]}. Reproducing sophisticated in vitro microenvironment usually takes days^{[18-20]} and the complexity of the microfluidic systems adversely affect the robustness of measurements^{[19]}.

To overcome the challenges of aforementioned approaches, we designed an assay for the dynamic profiling of antitumor activity of CAR-T cells. We employed micropatterning to precisely pattern multiple myeloma tumor cells into arrays of microscale islands^{[21]}. The tumor-cell islands have uniform size and shape and contain a similar number of tumor cells, facilitating the reproducibility of screening. Spatially segregating the tumor cells into microscale islands towards which CAR-T cells have to actively migrate allows the systematic study of trafficking and subsequent tumor killing. The arrays of tumor-cell islands are housed in customized microfluidic chambers which eliminates the drifting of CAR-T cells caused by convection-induced flow, ensuring robust cell interactions. We found that CAR T cells robustly migrate towards the tumor-cell islands, increasing the local effector-cell density and aggregating into large clusters that envelop the tumor cells and exert cytolytic effects on the tumor cells. The assay detects and quantifies differences in trafficking, clustering, and cytotoxicity of CAR T cells from different donors. Using the assay, we conducted multi-faceted characterizations of anti-BCMA and APRIL-based CAR T cell constructs against BCMA+ and BCMA knockout (KO) multiple myeloma MM.1s tumor cells. We demonstrate that APRIL-based CAR T cells efficiently clustered around and eliminated both tumor cell types, suggesting that this CAR T cell construct could reduce the incidence of antigen-negative escape. With the ease of use, the high throughput and reproducibility and the ability to dynamically map the antitumor activity of CAR T cells, the micropatterned tumor array (MiTA) is a useful tool for studying cancer immunology and aiding the pre-clinical evaluations of cell-based cancer immunotherapy.

Accordingly, provided herein are cell assay devices comprising a biocompatible substrate having an upper surface supporting a plurality of arrays of spots comprising an adhesion-promoting material; a biocompatible membrane having top and bottom surfaces and positioned adjacent to the upper surface of the substrate and defining a plurality of chambers within the membrane between the top surface and the bottom surface of the membrane, wherein the membrane comprises at least two openings in the top surface of the membrane into each chamber to provide access to the chambers; and wherein each chamber in the membrane is aligned with a respective one of the arrays of spots on the substrate; and a frame positioned on the top surface of the membrane, sandwiching the membrane between the frame and the substrate, wherein the frame defines a series of open-ended compartments, one compartment for each of the chambers within the membrane, wherein the compartments are in fluid communication with the chambers via the openings in the membrane.

In some embodiments, the cell assay device comprises a biocompatible substrate. Non-limiting examples of biocompatible substrates include glass, metal, composite, plastic, silicon, polymers, or other biocompatible or biologically unreactive (or biologically reactive) composition(s). In some embodiments, the substrate is planar or substantially planar. In some embodiments, the biocompatible substrate is a glass slide.

In some embodiments, the biocompatible substrate comprises a plurality of arrays of spots comprising an adhesion-promoting material.

Suitable adhesion-promoting materials include, but are not limited to, poly-L-Lysine, poly-D-Lysine, high-molecular-weight cationic copolymer of polyacrylamide and quaternized cationic monomer (e.g., ZETAG 8185, BASF), poly dopamine, collagen (e.g., type I), fibronectin, fibrin, gelatin, poly gelatin, extracellular matrix (ECM) proteins or peptides, ECM-like proteins or peptides, and combinations thereof (e.g., poly-L-lysine and high-molecular-weight cationic copolymer of polyacrylamide and quaternized cationic monomer (e.g., ZETAG 8185, BASF)). In some embodiments, the substrate can have a chemically modified surface (e.g., a silane such as (3-aminopropyl)triethoxy silane (APTES)) to promote adhesion, alone or in combination with other adhesion-promoting materials. In some embodiments, the substrate is coated with artificial adhesion-promoting polymers such as polyethylene glycol (PEG) and modified versions of PEG (e.g., PEG modified with an adhesion-promoting material such as a protein or peptide).

In some embodiments, the spots are circular in shape. In some embodiments, the spots are oval in shape. In some embodiments, the spots are toroidal in shape.

In some embodiments, the spots are between about 50 and about 500 μm in diameter. In some embodiments, the spots are between about 60 and about 500, about 70 and about 500, about 80 and about 500, about 90 and about 500, about 100 and about 500, about 110 and about 500, about 120 and about 500, about 130 and about 500, about 140 and about 500, about 150 and about 500, about 160 and about 500, about 170 and about 500, about 180 and about 500, about 190 and about 500, about 200 and about 500, about 210 and about 500, about 220 and about 500, about 230 and about 500, about 240 and about 500, about 250 and about 500, about 260 and about 500, about 270 and about 500, about 280 and about 500, about 290 and about 500, about 300 and about 500, about 310 and about 500, about 320 and about 500, about 330 and about 500, about 340 and about 500, about 350 and about 500, about 360 and about 500, about 370 and about 500, about 380 and about 500, about 390 and about 500, about 400 and about 500, about 410 and about 500, about 420 and about 500, about 430 and about 500, about 440 and about 500, about 450 and about 500, about 460 and about 500, about 470 and about 500, about 480 and about 500, about 490 and about 500, about 50 and about 490, about 60 and about 490, about 70 and about 490, about 80 and about 490, about 90 and about 490, about 100 and about 490, about 110 and about 490, about 120 and about 490, about 130 and about 490, about 140 and about 490, about 150 and about 490, about 160 and about 490, about 170 and about 490, about 180 and about 490, about 190 and about 490, about 200 and about 490, about 210 and about 490, about 220 and about 490, about 230 and about 490, about 240 and about 490, about 250 and about 490, about 260 and about 490, about 270 and about 490, about 280 and about 490, about 290 and about 490, about 300 and about 490, about 310 and about 490, about 320 and about 490, about 330 and about 490, about 340 and about 490, about 350 and about 490, about 360 and about 490, about 370 and about 490, about 380 and about 490, about 390 and about 490, about 400 and about 490, about 410 and about 490, about 420 and about 490, about 430 and about 490, about 440 and about 490, about 450 and about 490, about 460 and about 490, about 470 and about 490, about 480 and about 490, about 50 and about 480, about 60 and about 480, about 70 and about 480, about 80 and about 480, about 90 and about 480, about 100 and about 480, about 110 and about 480, about 120 and about 480, about 130 and about 480, about 140 and about 480, about 150 and about 480, about 160 and about 480, about 170 and about 480, about 180 and about 480, about 190 and about 480, about 200 and about 480, about 210 and about 480, about 220 and about 480, about 230 and about 480, about 240 and about 480, about 250 and about 480, about 260 and about 480, about 270 and about 480, about 280 and about 480, about 290 and about 480, about 300 and about 480, about 310 and about 480, about 320 and about 480, about 330 and about 480, about 340 and about 480, about 350 and about 480, about 360 and about 480, about 370 and about 480, about 380 and about 480, about 390 and about 480, about 400 and about 480, about 410 and about 480, about 420 and about 480, about 430 and about 480, about 440 and about 480, about 450 and about 480, about 460 and about 480, about 470 and about 480, about 50 and about 470, about 60 and about 470, about 70 and about 470, about 80 and about 470, about 90 and about 470, about 100 and about 470, about 110 and about 470, about 120 and about 470, about 130 and about 470, about 140 and about 470, about 150 and about 470, about 160 and about 470, about 170 and about 470, about 180 and about 470, about 190 and about 470, about 200 and about 470, about 210 and about 470, about 220 and about 470, about 230 and about 470, about 240 and about 470, about 250 and about 470, about 260 and about 470, about 270 and about 470, about 280 and about 470, about 290 and about 470, about 300 and about 470, about 310 and about 470, about 320 and about 470, about 330 and about 470, about 340 and about 470, about 350 and about 470, about 360 and about 470, about 370 and about 470, about 380 and about 470, about 390 and about 470, about 400 and about 470, about 410 and about 470, about 420 and about 470, about 430 and about 470, about 440 and about 470, about 450 and about 470, about 460 and about 470, about 50 and about 460, about 60 and about 460, about 70 and about 460, about 80 and about 460, about 90 and about 460, about 100 and about 460, about 110 and about 460, about 120 and about 460, about 130 and about 460, about 140 and about 460, about 150 and about 460, about 160 and about 460, about 170 and about 460, about 180 and about 460, about 190 and about 460, about 200 and about 460, about 210 and about 460, about 220 and about 460, about 230 and about 460, about 240 and about 460, about 250 and about 460, about 260 and about 460, about 270 and about 460, about 280 and about 460, about 290 and about 460, about 300 and about 460, about 310 and about 460, about 320 and about 460, about 330 and about 460, about 340 and about 460, about 350 and about 460, about 360 and about 460, about 370 and about 460, about 380 and about 460, about 390 and about 460, about 400 and about 460, about 410 and about 460, about 420 and about 460, about 430 and about 460, about 440 and about 460, about 450 and about 460, about 50 and about 450, about 60 and about 450, about 70 and about 450, about 80 and about 450, about 90 and about 450, about 100 and about 450, about 110 and about 450, about 120 and about 450, about 130 and about 450, about 140 and about 450, about 150 and about 450, about 160 and about 450, about 170 and about 450, about 180 and about 450, about 190 and about 450, about 200 and about 450, about 210 and about 450, about 220 and about 450, about 230 and about 450, about 240 and about 450, about 250 and about 450, about 260 and about 450, about 270 and about 450, about 280 and about 450, about 290 and about 450, about 300 and about 450, about 310 and about 450, about 320 and about 450, about 330 and about 450, about 340 and about 450, about 350 and about 450, about 360 and about 450, about 370 and about 450, about 380 and about 450, about 390 and about 450, about 400 and about 450, about 410 and about 450, about 420 and about 450, about 430 and about 450, about 440 and about 450, about 50 and about 440, about 60 and about 440, about 70 and about 440, about 80 and about 440, about 90 and about 440, about 100 and about 440, about 110 and about 440, about 120 and about 440, about 130 and about 440, about 140 and about 440, about 150 and about 440, about 160 and about 440, about 170 and about 440, about 180 and about 440, about 190 and about 440, about 200 and about 440, about 210 and about 440, about 220 and about 440, about 230 and about 440, about 240 and about 440, about 250 and about 440, about 260 and about 440, about 270 and about 440, about 280 and about 440, about 290 and about 440, about 300 and about 440, about 310 and about 440, about 320 and about 440, about 330 and about 440, about 340 and about 440, about 350 and about 440, about 360 and about 440, about 370 and about 440, about 380 and about 440, about 390 and about 440, about 400 and about 440, about 410 and about 440, about 420 and about 440, about 430 and about 440, about 50 and about 430, about 60 and about 430, about 70 and about 430, about 80 and about 430, about 90 and about 430, about 100 and about 430, about 110 and about 430, about 120 and about 430, about 130 and about 430, about 140 and about 430, about 150 and about 430, about 160 and about 430, about 170 and about 430, about 180 and about 430, about 190 and about 430, about 200 and about 430, about 210 and about 430, about 220 and about 430, about 230 and about 430, about 240 and about 430, about 250 and about 430, about 260 and about 430, about 270 and about 430, about 280 and about 430, about 290 and about 430, about 300 and about 430, about 310 and about 430, about 320 and about 430, about 330 and about 430, about 340 and about 430, about 350 and about 430, about 360 and about 430, about 370 and about 430, about 380 and about 430, about 390 and about 430, about 400 and about 430, about 410 and about 430, about 420 and about 430, about 50 and about 420, about 60 and about 420, about 70 and about 420, about 80 and about 420, about 90 and about 420, about 100 and about 420, about 110 and about 420, about 120 and about 420, about 130 and about 420, about 140 and about 420, about 150 and about 420, about 160 and about 420, about 170 and about 420, about 180 and about 420, about 190 and about 420, about 200 and about 420, about 210 and about 420, about 220 and about 420, about 230 and about 420, about 240 and about 420, about 250 and about 420, about 260 and about 420, about 270 and about 420, about 280 and about 420, about 290 and about 420, about 300 and about 420, about 310 and about 420, about 320 and about 420, about 330 and about 420, about 340 and about 420, about 350 and about 420, about 360 and about 420, about 370 and about 420, about 380 and about 420, about 390 and about 420, about 400 and about 420, about 410 and about 420, about 50 and about 410, about 60 and about 410, about 70 and about 410, about 80 and about 410, about 90 and about 410, about 100 and about 410, about 110 and about 410, about 120 and about 410, about 130 and about 410, about 140 and about 410, about 150 and about 410, about 160 and about 410, about 170 and about 410, about 180 and about 410, about 190 and about 410, about 200 and about 410, about 210 and about 410, about 220 and about 410, about 230 and about 410, about 240 and about 410, about 250 and about 410, about 260 and about 410, about 270 and about 410, about 280 and about 410, about 290 and about 410, about 300 and about 410, about 310 and about 410, about 320 and about 410, about 330 and about 410, about 340 and about 410, about 350 and about 410, about 360 and about 410, about 370 and about 410, about 380 and about 410, about 390 and about 410, about 400 and about 410, about 50 and about 400, about 60 and about 400, about 70 and about 400, about 80 and about 400, about 90 and about 400, about 100 and about 400, about 110 and about 400, about 120 and about 400, about 130 and about 400, about 140 and about 400, about 150 and about 400, about 160 and about 400, about 170 and about 400, about 180 and about 400, about 190 and about 400, about 200 and about 400, about 210 and about 400, about 220 and about 400, about 230 and about 400, about 240 and about 400, about 250 and about 400, about 260 and about 400, about 270 and about 400, about 280 and about 400, about 290 and about 400, about 300 and about 400, about 310 and about 400, about 320 and about 400, about 330 and about 400, about 340 and about 400, about 350 and about 400, about 360 and about 400, about 370 and about 400, about 380 and about 400, about 390 and about 400, about 50 and about 390, about 60 and about 390, about 70 and about 390, about 80 and about 390, about 90 and about 390, about 100 and about 390, about 110 and about 390, about 120 and about 390, about 130 and about 390, about 140 and about 390, about 150 and about 390, about 160 and about 390, about 170 and about 390, about 180 and about 390, about 190 and about 390, about 200 and about 390, about 210 and about 390, about 220 and about 390, about 230 and about 390, about 240 and about 390, about 250 and about 390, about 260 and about 390, about 270 and about 390, about 280 and about 390, about 290 and about 390, about 300 and about 390, about 310 and about 390, about 320 and about 390, about 330 and about 390, about 340 and about 390, about 350 and about 390, about 360 and about 390, about 370 and about 390, about 380 and about 390, about 50 and about 380, about 60 and about 380, about 70 and about 380, about 80 and about 380, about 90 and about 380, about 100 and about 380, about 110 and about 380, about 120 and about 380, about 130 and about 380, about 140 and about 380, about 150 and about 380, about 160 and about 380, about 170 and about 380, about 180 and about 380, about 190 and about 380, about 200 and about 380, about 210 and about 380, about 220 and about 380, about 230 and about 380, about 240 and about 380, about 250 and about 380, about 260 and about 380, about 270 and about 380, about 280 and about 380, about 290 and about 380, about 300 and about 380, about 310 and about 380, about 320 and about 380, about 330 and about 380, about 340 and about 380, about 350 and about 380, about 360 and about 380, about 370 and about 380, about 50 and about 370, about 60 and about 370, about 70 and about 370, about 80 and about 370, about 90 and about 370, about 100 and about 370, about 110 and about 370, about 120 and about 370, about 130 and about 370, about 140 and about 370, about 150 and about 370, about 160 and about 370, about 170 and about 370, about 180 and about 370, about 190 and about 370, about 200 and about 370, about 210 and about 370, about 220 and about 370, about 230 and about 370, about 240 and about 370, about 250 and about 370, about 260 and about 370, about 270 and about 370, about 280 and about 370, about 290 and about 370, about 300 and about 370, about 310 and about 370, about 320 and about 370, about 330 and about 370, about 340 and about 370, about 350 and about 370, about 360 and about 370, about 50 and about 360, about 60 and about 360, about 70 and about 360, about 80 and about 360, about 90 and about 360, about 100 and about 360, about 110 and about 360, about 120 and about 360, about 130 and about 360, about 140 and about 360, about 150 and about 360, about 160 and about 360, about 170 and about 360, about 180 and about 360, about 190 and about 360, about 200 and about 360, about 210 and about 360, about 220 and about 360, about 230 and about 360, about 240 and about 360, about 250 and about 360, about 260 and about 360, about 270 and about 360, about 280 and about 360, about 290 and about 360, about 300 and about 360, about 310 and about 360, about 320 and about 360, about 330 and about 360, about 340 and about 360, about 350 and about 360, about 50 and about 350, about 60 and about 350, about 70 and about 350, about 80 and about 350, about 90 and about 350, about 100 and about 350, about 110 and about 350, about 120 and about 350, about 130 and about 350, about 140 and about 350, about 150 and about 350, about 160 and about 350, about 170 and about 350, about 180 and about 350, about 190 and about 350, about 200 and about 350, about 210 and about 350, about 220 and about 350, about 230 and about 350, about 240 and about 350, about 250 and about 350, about 260 and about 350, about 270 and about 350, about 280 and about 350, about 290 and about 350, about 300 and about 350, about 310 and about 350, about 320 and about 350, about 330 and about 350, about 340 and about 350, about 50 and about 340, about 60 and about 340, about 70 and about 340, about 80 and about 340, about 90 and about 340, about 100 and about 340, about 110 and about 340, about 120 and about 340, about 130 and about 340, about 140 and about 340, about 150 and about 340, about 160 and about 340, about 170 and about 340, about 180 and about 340, about 190 and about 340, about 200 and about 340, about 210 and about 340, about 220 and about 340, about 230 and about 340, about 240 and about 340, about 250 and about 340, about 260 and about 340, about 270 and about 340, about 280 and about 340, about 290 and about 340, about 300 and about 340, about 310 and about 340, about 320 and about 340, about 330 and about 340, about 50 and about 330, about 60 and about 330, about 70 and about 330, about 80 and about 330, about 90 and about 330, about 100 and about 330, about 110 and about 330, about 120 and about 330, about 130 and about 330, about 140 and about 330, about 150 and about 330, about 160 and about 330, about 170 and about 330, about 180 and about 330, about 190 and about 330, about 200 and about 330, about 210 and about 330, about 220 and about 330, about 230 and about 330, about 240 and about 330, about 250 and about 330, about 260 and about 330, about 270 and about 330, about 280 and about 330, about 290 and about 330, about 300 and about 330, about 310 and about 330, about 320 and about 330, about 50 and about 320, about 60 and about 320, about 70 and about 320, about 80 and about 320, about 90 and about 320, about 100 and about 320, about 110 and about 320, about 120 and about 320, about 130 and about 320, about 140 and about 320, about 150 and about 320, about 160 and about 320, about 170 and about 320, about 180 and about 320, about 190 and about 320, about 200 and about 320, about 210 and about 320, about 220 and about 320, about 230 and about 320, about 240 and about 320, about 250 and about 320, about 260 and about 320, about 270 and about 320, about 280 and about 320, about 290 and about 320, about 300 and about 320, about 310 and about 320, about 50 and about 310, about 60 and about 310, about 70 and about 310, about 80 and about 310, about 90 and about 310, about 100 and about 310, about 110 and about 310, about 120 and about 310, about 130 and about 310, about 140 and about 310, about 150 and about 310, about 160 and about 310, about 170 and about 310, about 180 and about 310, about 190 and about 310, about 200 and about 310, about 210 and about 310, about 220 and about 310, about 230 and about 310, about 240 and about 310, about 250 and about 310, about 260 and about 310, about 270 and about 310, about 280 and about 310, about 290 and about 310, about 300 and about 310, about 50 and about 300, about 60 and about 300, about 70 and about 300, about 80 and about 300, about 90 and about 300, about 100 and about 300, about 110 and about 300, about 120 and about 300, about 130 and about 300, about 140 and about 300, about 150 and about 300, about 160 and about 300, about 170 and about 300, about 180 and about 300, about 190 and about 300, about 200 and about 300, about 210 and about 300, about 220 and about 300, about 230 and about 300, about 240 and about 300, about 250 and about 300, about 260 and about 300, about 270 and about 300, about 280 and about 300, about 290 and about 300, about 50 and about 290, about 60 and about 290, about 70 and about 290, about 80 and about 290, about 90 and about 290, about 100 and about 290, about 110 and about 290, about 120 and about 290, about 130 and about 290, about 140 and about 290, about 150 and about 290, about 160 and about 290, about 170 and about 290, about 180 and about 290, about 190 and about 290, about 200 and about 290, about 210 and about 290, about 220 and about 290, about 230 and about 290, about 240 and about 290, about 250 and about 290, about 260 and about 290, about 270 and about 290, about 280 and about 290, about 50 and about 280, about 60 and about 280, about 70 and about 280, about 80 and about 280, about 90 and about 280, about 100 and about 280, about 110 and about 280, about 120 and about 280, about 130 and about 280, about 140 and about 280, about 150 and about 280, about 160 and about 280, about 170 and about 280, about 180 and about 280, about 190 and about 280, about 200 and about 280, about 210 and about 280, about 220 and about 280, about 230 and about 280, about 240 and about 280, about 250 and about 280, about 260 and about 280, about 270 and about 280, about 50 and about 270, about 60 and about 270, about 70 and about 270, about 80 and about 270, about 90 and about 270, about 100 and about 270, about 110 and about 270, about 120 and about 270, about 130 and about 270, about 140 and about 270, about 150 and about 270, about 160 and about 270, about 170 and about 270, about 180 and about 270, about 190 and about 270, about 200 and about 270, about 210 and about 270, about 220 and about 270, about 230 and about 270, about 240 and about 270, about 250 and about 270, about 260 and about 270, about 50 and about 260, about 60 and about 260, about 70 and about 260, about 80 and about 260, about 90 and about 260, about 100 and about 260, about 110 and about 260, about 120 and about 260, about 130 and about 260, about 140 and about 260, about 150 and about 260, about 160 and about 260, about 170 and about 260, about 180 and about 260, about 190 and about 260, about 200 and about 260, about 210 and about 260, about 220 and about 260, about 230 and about 260, about 240 and about 260, about 250 and about 260, about 50 and about 250, about 60 and about 250, about 70 and about 250, about 80 and about 250, about 90 and about 250, about 100 and about 250, about 110 and about 250, about 120 and about 250, about 130 and about 250, about 140 and about 250, about 150 and about 250, about 160 and about 250, about 170 and about 250, about 180 and about 250, about 190 and about 250, about 200 and about 250, about 210 and about 250, about 220 and about 250, about 230 and about 250, about 240 and about 250, about 50 and about 240, about 60 and about 240, about 70 and about 240, about 80 and about 240, about 90 and about 240, about 100 and about 240, about 110 and about 240, about 120 and about 240, about 130 and about 240, about 140 and about 240, about 150 and about 240, about 160 and about 240, about 170 and about 240, about 180 and about 240, about 190 and about 240, about 200 and about 240, about 210 and about 240, about 220 and about 240, about 230 and about 240, about 50 and about 230, about 60 and about 230, about 70 and about 230, about 80 and about 230, about 90 and about 230, about 100 and about 230, about 110 and about 230, about 120 and about 230, about 130 and about 230, about 140 and about 230, about 150 and about 230, about 160 and about 230, about 170 and about 230, about 180 and about 230, about 190 and about 230, about 200 and about 230, about 210 and about 230, about 220 and about 230, about 50 and about 220, about 60 and about 220, about 70 and about 220, about 80 and about 220, about 90 and about 220, about 100 and about 220, about 110 and about 220, about 120 and about 220, about 130 and about 220, about 140 and about 220, about 150 and about 220, about 160 and about 220, about 170 and about 220, about 180 and about 220, about 190 and about 220, about 200 and about 220, about 210 and about 220, about 50 and about 210, about 60 and about 210, about 70 and about 210, about 80 and about 210, about 90 and about 210, about 100 and about 210, about 110 and about 210, about 120 and about 210, about 130 and about 210, about 140 and about 210, about 150 and about 210, about 160 and about 210, about 170 and about 210, about 180 and about 210, about 190 and about 210, about 200 and about 210, about 50 and about 200, about 60 and about 200, about 70 and about 200, about 80 and about 200, about 90 and about 200, about 100 and about 200, about 110 and about 200, about 120 and about 200, about 130 and about 200, about 140 and about 200, about 150 and about 200, about 160 and about 200, about 170 and about 200, about 180 and about 200, about 190 and about 200, about 50 and about 190, about 60 and about 190, about 70 and about 190, about 80 and about 190, about 90 and about 190, about 100 and about 190, about 110 and about 190, about 120 and about 190, about 130 and about 190, about 140 and about 190, about 150 and about 190, about 160 and about 190, about 170 and about 190, about 180 and about 190, about 50 and about 180, about 60 and about 180, about 70 and about 180, about 80 and about 180, about 90 and about 180, about 100 and about 180, about 110 and about 180, about 120 and about 180, about 130 and about 180, about 140 and about 180, about 150 and about 180, about 160 and about 180, about 170 and about 180, about 50 and about 170, about 60 and about 170, about 70 and about 170, about 80 and about 170, about 90 and about 170, about 100 and about 170, about 110 and about 170, about 120 and about 170, about 130 and about 170, about 140 and about 170, about 150 and about 170, about 160 and about 170, about 50 and about 160, about 60 and about 160, about 70 and about 160, about 80 and about 160, about 90 and about 160, about 100 and about 160, about 110 and about 160, about 120 and about 160, about 130 and about 160, about 140 and about 160, about 150 and about 160, about 50 and about 150, about 60 and about 150, about 70 and about 150, about 80 and about 150, about 90 and about 150, about 100 and about 150, about 110 and about 150, about 120 and about 150, about 130 and about 150, about 140 and about 150, about 50 and about 140, about 60 and about 140, about 70 and about 140, about 80 and about 140, about 90 and about 140, about 100 and about 140, about 110 and about 140, about 120 and about 140, about 130 and about 140, about 50 and about 130, about 60 and about 130, about 70 and about 130, about 80 and about 130, about 90 and about 130, about 100 and about 130, about 110 and about 130, about 120 and about 130, about 50 and about 120, about 60 and about 120, about 70 and about 120, about 80 and about 120, about 90 and about 120, about 100 and about 120, about 110 and about 120, about 50 and about 110, about 60 and about 110, about 70 and about 110, about 80 and about 110, about 90 and about 110, about 100 and about 110, about 50 and about 100, about 60 and about 100, about 70 and about 100, about 80 and about 100, about 90 and about 100, about 50 and about 90 about 60 and about 90, about 70 and about 90, about 80 and about 90, about 50 and about 80 about 60 and about 80, about 70 and about 80, about 50 and about 70 about 60 and about 70, or about 50 and about 60 μm in diameter.

In some embodiments, the total number of spots is between about 1 and about 5,000. In some embodiments, the total number of spots is between about 500 and about 5,000, about 1,000 and about 5,000, about 1,500 and about 5,000, about 2,000 and about 5,000, about 2,500 and about 5,000, about 3,000 and about 5,000, about 3,500 and about 5,000, about 4,000 and about 5,000, about 4,500 and about 5,000, about 500 and about 4,500, about 1,000 and about 4,500, about 1,500 and about 4,500, about 2,000 and about 4,500, about 2,500 and about 4,500, about 3,000 and about 4,500, about 3,500 and about 4,500, about 4,000 and about 4,500, about 500 and about 4,000, about 1,000 and about 4,000, about 1,500 and about 4,000, about 2,000 and about 4,000, about 2,500 and about 4,000, about 3,000 and about 4,000, about 3,500 and about 4,000, about 500 and about 3,500, about 1,000 and about 3,500, about 1,500 and about 3,500, about 2,000 and about 3,500, about 2,500 and about 3,500, about 3,000 and about 3,500, about 500 about 500 and about 3,000, about 1,000 and about 3,000, about 1,500 and about 3,000, about 2,000 and about 3,000, about 2,500 and about 3,000, about 500 and about 2,500, about 1,000 and about 2,500, about 1,500 and about 2,500, about 2,000 and about 2,500, about 500 to about 2,000, about 1,000 to about 2,000, about 1,500 to about 2,000, about 500 to about 1,500, about 1,000 to about 1,500, about 500 to about 1,000, or about 1 to about 500. In some embodiments, the total number of spots is about 4,096.

In some embodiments, the number of spots in each array is between about 1 and about 100. In some embodiments, the number of spots on each array is between about 4 and about 100. In some embodiments, the number of spots on each array is between about 9 and about 100, about 16 and about 100, about 25 and about 100, about 36 and about 100, about 49 and about 100, about 64 and about 100, about 81 and about 100, or about 1 and about 4. In some embodiments, the number of spots on each array is about 64.

In some embodiments, the distance between spots on an array is at least about 35 μm. In some embodiments, the distance between spots on an array is at least about 20, about 25, about 30, about 35, about 40, about 45, about 50, about 55, about 60, about 65, about 70, about 75, about 80, about 85, about 90, about 95, or about 100 μm.

In some embodiments, the cell assay device comprises a biocompatible membrane. In some embodiments, the biocompatible membrane comprises a polymer. In some embodiments, the polymer comprises one or more of a polydimethylsiloxane, a plastic, or a hydrogel.

In some embodiments, the membrane is permeable. In some embodiments, the membrane is impermeable. In some embodiments, the membrane is semi-permeable. In some embodiments, the membrane is permeable for molecules and particles having a diameter of from about 1 nm to about 100 μm. In some embodiments, the membrane is gas permeable. In some embodiments, the membrane is gas impermeable. In some embodiments, the membrane is liquid permeable. In some embodiments, the membrane is liquid impermeable.

In some embodiments, the membrane has a thickness of about 1.5 mm. In some embodiments, the membrane has a thickness of from about 0.1 to about 3, about 0.2 to about 3, about 0.3 to about 3, about 0.4 to about 3, about 0.5 to about 3, about 0.6 to about 3, about 0.7 to about 3, about 0.8 to about 3, about 0.9 to about 3, about 1 to about 3, about 1.1 to about 3, about 1.2 to about 3, about 1.3 to about 3, about 1.4 to about 3, about 1.5 to about 3, about 1.6 to about 3, about 1.7 to about 3, about 1.8 to about 3, about 1.9 to about 3, about 2 to about 3, about 2.1 to about 3, about 2.2 to about 3, about 2.3 to about 3, about 2.4 to about 3, about 2.5 to about 3, about 2.6 to about 3, about 2.7 to about 3, about 2.8 to about 3, about 2.9 to about 3, about 0.1 to about 2.9, about 0.2 to about 2.9, about 0.3 to about 2.9, about 0.4 to about 2.9 about 0.5 to about 2.9, about 0.6 to about 2.9, about 0.7 to about 2.9, about 0.8 to about 2.9, about 0.9 to about 2.9, about 1 to about 2.9, about 1.1 to about 2.9, about 1.2 to about 2.9, about 1.3 to about 2.9, about 1.4 to about 2.9, about 1.5 to about 2.9, about 1.6 to about 2.9, about 1.7 to about 2.9, about 1.8 to about 2.9, about 1.9 to about 2.9, about 2 to about 2.9, about 2.1 to about 2.9, about 2.2 to about 2.9, about 2.3 to about 2.9, about 2.4 to about 2.9, about 2.5 to about 2.9, about 2.6 to about 2.9, about 2.7 to about 2.9, about 2.8 to about 2.9, about 0.1 to about 2.8, about 0.2 to about 2.8, about 0.3 to about 2.8, about 0.4 to about 2.8 about 0.5 to about 2.8, about 0.6 to about 2.8, about 0.7 to about 2.8, about 0.8 to about 2.8, about 0.9 to about 2.8, about 1 to about 2.8, about 1.1 to about 2.8, about 1.2 to about 2.8, about 1.3 to about 2.8, about 1.4 to about 2.8, about 1.5 to about 2.8, about 1.6 to about 2.8, about 1.7 to about 2.8, about 1.8 to about 2.8, about 1.9 to about 2.8, about 2 to about 2.8, about 2.1 to about 2.8, about 2.2 to about 2.8, about 2.3 to about 2.8, about 2.4 to about 2.8, about 2.5 to about 2.8, about 2.6 to about 2.8, about 2.7 to about 2.8, about 0.1 to about 2.7, about 0.2 to about 2.7, about 0.3 to about 2.7, about 0.4 to about 0.5, about 0.5 to about 2.7, about 0.6 to about 2.7, about 0.7 to about 2.7, about 0.8 to about 2.7, about 0.9 to about 2.7, about 1 to about 2.7, about 1.1 to about 2.7, about 1.2 to about 2.7, about 1.3 to about 2.7, about 1.4 to about 2.7, about 1.5 to about 2.7, about 1.6 to about 2.7, about 1.7 to about 2.7, about 1.8 to about 2.7, about 1.9 to about 2.7, about 2 to about 2.7, about 2.1 to about 2.7, about 2.2 to about 2.7, about 2.3 to about 2.7, about 2.4 to about 2.7, about 2.5 to about 2.7, about 2.6 to about 2.7, about 0.1 to about 2.6, about 0.2 to about 2.6, about 0.3 to about 2.6, about 0.4 to about 0.5, about 0.5 to about 2.6, about 0.6 to about 2.6, about 0.7 to about 2.6, about 0.8 to about 2.6, about 0.9 to about 2.6, about 1 to about 2.6, about 1.1 to about 2.6, about 1.2 to about 2.6, about 1.3 to about 2.6, about 1.4 to about 2.6, about 1.5 to about 2.6, about 1.6 to about 2.6, about 1.7 to about 2.6, about 1.8 to about 2.6, about 1.9 to about 2.6, about 2 to about 2.6, about 2.1 to about 2.6, about 2.2 to about 2.6, about 2.3 to about 2.6, about 2.4 to about 2.6, about 2.5 to about 2.6, about 0.1 to about 2.5, about 0.2 to about 2.5, about 0.3 to about 2.5, about 0.4 to about 0.5, about 0.5 to about 2.5, about 0.6 to about 2.5, about 0.7 to about 2.5, about 0.8 to about 2.5, about 0.9 to about 2.5, about 1 to about 2.5, about 1.1 to about 2.5, about 1.2 to about 2.5, about 1.3 to about 2.5, about 1.4 to about 2.5, about 1.5 to about 2.5, about 1.6 to about 2.5, about 1.7 to about 2.5, about 1.8 to about 2.5, about 1.9 to about 2.5, about 2 to about 2.5, about 2.1 to about 2.5, about 2.2 to about 2.5, about 2.3 to about 2.5, about 2.4 to about 2.5, about 0.1 to about 2.4, about 0.2 to about 2.4, about 0.3 to about 2.4, about 0.4 to about 0.5, about 0.5 to about 2.4, about 0.6 to about 2.4, about 0.7 to about 2.4, about 0.8 to about 2.4, about 0.9 to about 2.4, about 1 to about 2.4, about 1.1 to about 2.4, about 1.2 to about 2.4, about 1.3 to about 2.4, about 1.4 to about 2.4, about 1.5 to about 2.4, about 1.6 to about 2.4, about 1.7 to about 2.4, about 1.8 to about 2.4, about 1.9 to about 2.4, about 2 to about 2.4, about 2.1 to about 2.4, about 2.2 to about 2.4, about 2.3 to about 2.4, about 0.1 to about 2.3, about 0.2 to about 2.3, about 0.3 to about 2.3, about 0.4 to about 0.5, about 0.5 to about 2.3, about 0.6 to about 2.3, about 0.7 to about 2.3, about 0.8 to about 2.3, about 0.9 to about 2.3, about 1 to about 2.3, about 1.1 to about 2.3, about 1.2 to about 2.3, about 1.3 to about 2.3, about 1.4 to about 2.3, about 1.5 to about 2.3, about 1.6 to about 2.3, about 1.7 to about 2.3, about 1.8 to about 2.3, about 1.9 to about 2.3, about 2 to about 2.3, about 2.1 to about 2.3, about 2.2 to about 2.3, about 0.1 to about 2.2, about 0.2 to about 2.2, about 0.3 to about 2.2, about 0.4 to about 0.5, about 0.5 to about 2.2, about 0.6 to about 2.2, about 0.7 to about 2.2, about 0.8 to about 2.2, about 0.9 to about 2.2, about 1 to about 2.2, about 1.1 to about 2.2, about 1.2 to about 2.2, about 1.3 to about 2.2, about 1.4 to about 2.2, about 1.5 to about 2.2, about 1.6 to about 2.2, about 1.7 to about 2.2, about 1.8 to about 2.2, about 1.9 to about 2.2, about 2 to about 2.2, about 2.1 to about 2.2, about 0.1 to about 2.1, about 0.2 to about 2.1, about 0.3 to about 2.1, about 0.4 to about 0.5, about 0.5 to about 2.1, about 0.6 to about 2.1, about 0.7 to about 2.1, about 0.8 to about 2.1, about 0.9 to about 2.1, about 1 to about 2.1, about 1.1 to about 2.1, about 1.2 to about 2.1, about 1.3 to about 2.1, about 1.4 to about 2.1, about 1.5 to about 2.1, about 1.6 to about 2.1, about 1.7 to about 2.1, about 1.8 to about 2.1, about 1.9 to about 2.1, about 2 to about 2.1, about 0.1 to about 2, about 0.2 to about 2, about 0.3 to about 2, about 0.4 to about 0.5, about 0.5 to about 2, about 0.6 to about 2, about 0.7 to about 2, about 0.8 to about 2, about 0.9 to about 2, about 1 to about 2, about 1.1 to about 2, about 1.2 to about 2, about 1.3 to about 2, about 1.4 to about 2, about 1.5 to about 2, about 1.6 to about 2, about 1.7 to about 2, about 1.8 to about 2, about 1.9 to about 2, about 0.1 to about 1.9, about 0.2 to about 1.9, about 0.3 to about 1.9, about 0.4 to about 0.5, about 0.5 to about 1.9, about 0.6 to about 1.9, about 0.7 to about 1.9, about 0.8 to about 1.9, about 0.9 to about 1.9, about 1 to about 1.9, about 1.1 to about 1.9, about 1.2 to about 1.9, about 1.3 to about 1.9, about 1.4 to about 1.9, about 1.5 to about 1.9, about 1.6 to about 1.9, about 1.7 to about 1.9, about 1.8 to about 1.9, about 0.1 to about 1.8, about 0.2 to about 1.8, about 0.3 to about 1.8, about 0.4 to about 0.5, about 0.5 to about 1.8, about 0.6 to about 1.8, about 0.7 to about 1.8, about 0.8 to about 1.8, about 0.9 to about 1.8, about 1 to about 1.8, about 1.1 to about 1.8, about 1.2 to about 1.8, about 1.3 to about 1.8, about 1.4 to about 1.8, about 1.5 to about 1.8, about 1.6 to about 1.8, about 1.7 to about 1.8, about 0.1 to about 1.7, about 0.2 to about 1.7, about 0.3 to about 1.7, about 0.4 to about 0.5, about 0.5 to about 1.7, about 0.6 to about 1.7, about 0.7 to about 1.7, about 0.8 to about 1.7, about 0.9 to about 1.7, about 1 to about 1.7, about 1.1 to about 1.7, about 1.2 to about 1.7, about 1.3 to about 1.7, about 1.4 to about 1.7, about 1.5 to about 1.7, about 1.6 to about 1.7, about 0.1 to about 1.6, about 0.2 to about 1.6, about 0.3 to about 1.6, about 0.4 to about 0.5, about 0.5 to about 1.6, about 0.6 to about 1.6, about 0.7 to about 1.6, about 0.8 to about 1.6, about 0.9 to about 1.6, about 1 to about 1.6, about 1.1 to about 1.6, about 1.2 to about 1.6, about 1.3 to about 1.6, about 1.4 to about 1.6, about 1.5 to about 1.6, about 0.1 to about 1.5, about 0.2 to about 1.5, about 0.3 to about 1.5, about 0.4 to about 0.5, about 0.5 to about 1.5, about 0.6 to about 1.5, about 0.7 to about 1.5, about 0.8 to about 1.5, about 0.9 to about 1.5, about 1 to about 1.5, about 1.1 to about 1.5, about 1.2 to about 1.5, about 1.3 to about 1.5, about 1.4 to about 1.5, about 0.1 to about 1.4, about 0.2 to about 1.4, about 0.3 to about 1.4, about 0.4 to about 0.5, about 0.5 to about 1.4, about 0.6 to about 1.4, about 0.7 to about 1.4, about 0.8 to about 1.4, about 0.9 to about 1.4, about 1 to about 1.4, about 1.1 to about 1.4, about 1.2 to about 1.4, about 1.3 to about 1.4, about 0.1 to about 1.3, about 0.2 to about 1.3, about 0.3 to about 1.3, about 0.4 to about 0.5, about 0.5 to about 1.3, about 0.6 to about 1.3, about 0.7 to about 1.3, about 0.8 to about 1.3, about 0.9 to about 1.3, about 1 to about 1.3, about 1.1 to about 1.3, about 1.2 to about 1.3, about 0.1 to about 1.2, about 0.2 to about 1.2, about 0.3 to about 1.2, about 0.4 to about 0.5, about 0.5 to about 1.2, about 0.6 to about 1.2, about 0.7 to about 1.2, about 0.8 to about 1.2, about 0.9 to about 1.2, about 1 to about 1.2, about 1.1 to about 1.2, about 0.1 to about 1.1, about 0.2 to about 1.1, about 0.3 to about 1.1, about 0.4 to about 0.5, about 0.5 to about 1.1, about 0.6 to about 1.1, about 0.7 to about 1.1, about 0.8 to about 1.1, about 0.9 to about 1.1, about 1 to about 1.1, about 0.1 to about 1, about 0.2 to about 1, about 0.3 to about 1, about 0.4 to about 0.5, about 0.5 to about 1, about 0.6 to about 1, about 0.7 to about 1, about 0.8 to about 1, about 0.9 to about 1, about 0.1 to about 0.9, about 0.2 to about 0.9, about 0.3 to about 0.9, about 0.4 to about 0.5, about 0.5 to about 0.9, about 0.6 to about 0.9, about 0.7 to about 0.9, about 0.8 to about 0.9, about 0.1 to about 0.8, about 0.2 to about 0.8, about 0.3 to about 0.8, about 0.4 to about 0.5, about 0.5 to about 0.8, about 0.6 to about 0.8, about 0.7 to about 0.8, about 0.1 to about 0.7, about 0.2 to about 0.7, about 0.3 to about 0.7, about 0.4 to about 0.5, about 0.5 to about 0.7, about 0.6 to about 0.7, about 0.1 to about 0.6, about 0.2 to about 0.6, about 0.3 to about 0.6, about 0.4 to about 0.5, about 0.5 to about 0.6, about 0.1 to about 0.5, about 0.2 to about 0.5, about 0.3 to about 0.5, about 0.4 to about 0.5, about 0.1 to about 0.4, about 0.2 to about 0.4, about 0.3 to about 0.4, about 0.1 to about 0.3, about 0.2 to about 0.3, or about 0.1 to about 0.2 mm.

In some embodiments, the membrane defines a plurality of chambers within the membrane between the top surface and the bottom surface of the membrane.

In some embodiments, the height of the chamber is between about 50 to about 150 μm. In some embodiments, the height of the chamber is between about 20 and about 200, about 30 and about 200, about 40 and about 200, about 50 and about 200, about 60 and about 200, about 70 and about 200, about 80 and about 200, about 90 and about 200, about 100 and about 200, about 110 and about 200, about 120 and about 200, about 130 and about 200, about 140 and about 200, about 150 and about 200, about 160 and about 200, about 170 and about 200, about 180 and about 200, about 190 and about 200, about 20 and about 190, about 30 and about 190, about 40 and about 190, about 50 and about 190, about 60 and about 190, about 70 and about 190, about 80 and about 190, about 90 and about 190, about 100 and about 190, about 110 and about 190, about 120 and about 190, about 130 and about 190, about 140 and about 190, about 150 and about 190, about 160 and about 190, about 170 and about 190, about 180 and about 190, about 20 and about 180, about 30 and about 180, about 40 and about 180, about 50 and about 180, about 60 and about 180, about 70 and about 180, about 80 and about 180, about 90 and about 180, about 100 and about 180, about 110 and about 180, about 120 and about 180, about 130 and about 180, about 140 and about 180, about 150 and about 180, about 160 and about 180, about 170 and about 180, about 20 and about 170, about 30 and about 170, about 40 and about 170, about 50 and about 170, about 60 and about 170, about 70 and about 170, about 80 and about 170, about 90 and about 170, about 100 and about 170, about 110 and about 170, about 120 and about 170, about 130 and about 170, about 140 and about 170, about 150 and about 170, about 160 and about 170, about 20 and about 160, about 30 and about 160, about 40 and about 160, about 50 and about 160, about 60 and about 160, about 70 and about 160, about 80 and about 160, about 90 and about 160, about 100 and about 160, about 110 and about 160, about 120 and about 160, about 130 and about 160, about 140 and about 160, about 150 and about 160, about 20 and about 150, about 30 and about 150, about 40 and about 150, about 50 and about 150, about 60 and about 150, about 70 and about 150, about 80 and about 150, about 90 and about 150, about 100 and about 150, about 110 and about 150, about 120 and about 150, about 130 and about 150, about 140 and about 150, about 20 and about 140, about 30 and about 140, about 40 and about 140, about 50 and about 140, about 60 and about 140, about 70 and about 140, about 80 and about 140, about 90 and about 140, about 100 and about 140, about 110 and about 140, about 120 and about 140, about 130 and about 140, about 20 and about 130, about 30 and about 130, about 40 and about 130, about 50 and about 130, about 60 and about 130, about 70 and about 130, about 80 and about 130, about 90 and about 130, about 100 and about 130, about 110 and about 130, about 120 and about 130, about 20 and about 120, about 30 and about 120, about 40 and about 120, about 50 and about 120, about 60 and about 120, about 70 and about 120, about 80 and about 120, about 90 and about 120, about 100 and about 120, about 110 and about 120, about 20 and about 110, about 30 and about 110, about 40 and about 110, about 50 and about 110, about 60 and about 110, about 70 and about 110, about 80 and about 110, about 90 and about 110, about 100 and about 110, about 20 and about 100, about 30 and about 100, about 40 and about 100, about 50 and about 100, about 60 and about 100, about 70 and about 100, about 80 and about 100, about 90 and about 100, about 20 and about 90, about 30 and about 90, about 40 and about 90, about 50 and about 90, about 60 and about 90, about 70 and about 90, about 80 and about 90, about 20 and about 80, about 30 and about 80, about 40 and about 80, about 50 and about 80, about 60 and about 80, about 70 and about 80, about 20 and about 70, about 30 and about 70, about 40 and about 70, about 50 and about 70, about 60 and about 70, about 20 and about 60, about 30 and about 60, about 40 and about 60, about 50 and about 60, about 20 and about 50, about 30 and about 50, about 40 and about 50, about 20 and about 40, about 30 and about 40, or about 20 and about 30 μm.

In some embodiments, the membrane comprises at least two openings in the top surface of the membrane into each chamber to provide access to the chambers. In some embodiments, the membrane comprises 2, 3, 4, 5, 6, 7, 8, 9, 10, 11, 12, 13, 14, 15, 16, 17, 18, 19, or 20 openings in the top surface of the membrane into each chamber to provide access to the chambers, e.g., at least 2 and as many as 20 openings.

The chamber can be any three dimensional shape. In some embodiments, the chamber is square. In some embodiments, the chamber is rectangular. In some embodiments, the chamber is round. In some embodiments, the chamber length and width are about 6.4 mm. In some embodiments, the chamber length and width are about 3.7 mm.

The openings in the top surface of the membrane can be placed so that when fluid (e.g. fluid comprising immune cells) is added to the chamber in one of the openings, fluid within the chamber is displaced into the compartment through another of the opening(s).

The membranes described herein can be manufactured using various methods. In one example, membranes described herein are fabricated using standard photolithography or soft lithography techniques to generate a silicon wafer, which is used as a negative mold to generate polydimethylsiloxane (PDMS) membrane(s). For example, the mold can be formed by applying and sequentially patterning two layers of photoresist (e.g., SUB, Microchem, Newton, Mass.) on a silicon wafer using two photolithography masks according to known methods. The masks can contain features that define the different aspects of the membrane such as the chamber.

The wafer with the patterned photoresist then can be used as a master mold to form the membranes. A PDMS (e.g., Fisher Scientific, Fair Lawn, N.J.) solution then is applied to the master mold and cured. After curing, the PDMS layer solidifies and can be peeled off the master mold. The solidified PDMS layer includes grooves and/or recesses corresponding to the chamber of the membrane. In some implementations, the mold pattern is designed to include the features of multiple membranes.

Each membrane can be cut out from the PDMS layer. The openings in the top surface of the membrane can be formed, for example, by using a hole puncher to punch out PDMS material from the PDMS layer. A bottom surface of the PDMS devices can be plasma treated to enhance the bonding properties of the PDMS, and can be heated to induce bonding with the substrate. The membrane can also be exposed to plasma treatment prior to bonding to render the chambers hydrophilic.

In some embodiments, the cell assay device comprises a frame positioned on the top surface of the membrane, sandwiching the membrane between the frame and the substrate, wherein the frame defines a series of open-ended compartments, one compartment for each of the chambers within the membrane, wherein the compartments are in fluid communication with the chambers via the openings in the membrane.

In some embodiments, the frame is rigid.

In some embodiments, the compartment length and width is about 9.6 mm. In some embodiments, the compartment is about 9.6 mm square. In some embodiments, the compartment length and width is about 7 mm. In some embodiments, the compartment is about 7 mm square. In some embodiments the compartment is about 7 mm×7 mm×300 μm.

Also provided herein are methods of assaying the activity of immune cells on target cells. The methods make use of the micropatterning of target cells on the devices of the disclosure for high throughput and reproducible mapping of the activity of immune cells on target cells. Thus, the methods of assaying the activity of immune cells on target cells provided herein are useful, for example, in detecting clinically relevant interactions between immune cells and target cells and provide an important tool for pre-clinical evaluations, e.g., of cell-based cancer immunotherapy.

In the methods provided herein, target cells are immobilized on the device, while immune cells are introduced into the chamber of the device in, for example, a liquid suspension and allowed to migrate towards the target cells. The interaction between the target cells and immune cells can be monitored in real time, including, for example, by time-lapse microscopy. Thus, interactions such as, for example, trafficking, clustering, and cytotoxicity, can be measured comprehensively.

Thus, in one embodiment, the method of assaying the activity of immune cells on target cells comprises introducing the target cells into the chamber of a device of the disclosure through one of the at least two membrane opening; permitting the introduced tumor cells to settle onto the adhesive dots and adhere thereto; flushing the device to remove non-adherent tumor cells; introducing an extracellular matrix protein into the chamber of the device; filling the chamber of the device with media; introducing the immune cells into the chamber of the device; and imaging the chamber.

In some embodiments, the immune cells comprise T cells, natural killer cells, B cells, neutrophils, eosinophils, dendritic cells, macrophages, mast cells, basophils, or a combination thereof. In some embodiments, the immune cells are T cells. In some embodiments, the T cells cells are chimeric antigen receptor T cells (CAR T cells).

In some embodiments, the target cell is a tumor cell. In some embodiments, the target cell is a cancer cell. In some embodiments, the target cell is a cancer tumor cell.

Non-limiting examples of cancer cells include acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, Wilms tumor, and combinations thereof.

Non-limiting examples of cancer tumor cells include fibrosarcoma, myxosarcoma, liposarcoma, chondrosarcoma, osteosarcoma, chordoma, malignant fibrous histiocytoma, hemangiosarcoma, angiosarcoma, lymphangiosarcoma, mesothelioma, leukemia, plasmocytoma, multiple myeloma, Hodgkin lymphoma, Non-Hodgkin lymphoma, leiomyosarcoma, rhabdomyosarcoma, squamous cell carcinoma, epidermoid carcinoma, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma, glioblastoma, neuroblastoma, medulloblastoma, malignant meningioma, malignant schwannoma, neurofibrosarcoma, parathyroid carcinoma, medullary carcinoma of thyroid, bronchial carcinoid, oat cell carcinoma, malignant pheochromocytoma, islet cell carcinoma, malignant carcinoid, malignant paraganglioma, melanoma, Merkel cell neoplasm, cytosarcoma phylloides, Wilms tumor, seminoma, dysgerminoma, endodermal sinus tumor, teratocarcinoma, Sertoli-Leydig cell tumor, granulose-theca cell tumor, hilar cell tumor, lipid cell tumor, and combinations thereof.

In some embodiments, the target cells comprise a reporter gene. In some embodiments, the immune cells comprise a reporter gene. In some embodiments, the reporter gene is a gene that encodes a fluorescent protein. In some embodiments, the reporter gene encodes a green fluorescent protein, a red fluorescent protein, a yellow fluorescent protein, a blue fluorescent protein, a cyan fluorescent protein, an orange fluorescent protein, or combinations thereof.

In some embodiments, reporter gene encodes a green fluorescent protein selected from GFP, EGFP, Emerland, Superfold GFP, Azami Green, mWasabi, TagGFP, TurboGFP, AcGFP, ZsGreen, T-Sapphire, click beetle green, and combinations thereof.

In some embodiments, the reporter gene encodes a blue fluorescent protein selected from EBFP, EBFP2, Azurite, mTagBFP, and combinations thereof.

In some embodiments, the reporter gene encodes a cyan fluorescent protein selected from ECFP, mECFP, Cerulean, mTurqoise, CyPet, AmCyan1, Midori-Ishi Cyan, TagCFP, mTFP11 (Teal), and combinations thereof.

In some embodiments, the reporter gene encodes a yellow fluorescent protein selected from EYFP, Topaz, Venus, mCitrine, YPet, TagYFP, PhiYFP, ZsYellow1, mBanana, and combinations thereof.

In some embodiments, the reporter gene encodes an orange fluorescent protein selected from Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, and combinations thereof.

In some embodiments, the reporter gene encodes a red fluorescent protein selected from mRuby, mApple, mStrawberry, AsRed2, mRFP1, JRed, mCherry, HcRed1, mRaspberry, dKeima-Tandem, HcRed-Tandem, mPlum, AQ143, or combinations thereof.

In some embodiments, the reporter gene is a transgene. In some embodiments, the reporter gene is expressed constitutively.

In some embodiments, the tumor cells are introduced into the chamber of a device described herein through one of the at least two membrane openings. In some embodiments, the tumor cells are introduced into the chamber of a device described herein through more than one of the at least two membrane openings.

In some embodiments, between about 2.5 million and about 7.5 million tumor cells are introduced into the chamber. In some embodiments, between about 2 million and about 8 million, about 3 million and about 8 million, about 4 million and about 8 million, about 5 million and about 8 million, about 6 million and about 8 million, about 7 million and about 8 million, about 2 million and about 7 million, about 3 million and about 7 million, about 4 million and about 7 million, about 5 million and about 7 million, about 6 million and about 7 million, about 2 million and about 6 million, about 3 million and about 6 million, about 4 million and about 6 million, about 5 million and about 6 million, about 2 million and about 5 million, about 3 million and about 5 million, about 4 million and about 5 million, about 2 million and about 4 million, or about 2 million and about 3 million tumor cells are introduced into the compartment.

In some embodiments, the cells are in contact with the device for about 1, about 2, about 3, about 4, about 5, about 6, about 7, about 8, about 9, about 10, about 15, about 20, about 25, or about 30 minutes prior to flushing.

In some embodiments, cells that are not adhered to the spots of the device are removed by flushing the chamber. In some embodiments, the chamber is flushed with fresh media. Suitable media include any cell-culture media compatible with the target cells, including, but not limited to, serum-free media formulations. In some embodiments, the media is Dulbecco's Modified Eagle Medium (DMEM), Iscove's Modified Dulbecco's Medium (IMDM), RPMI 1640 Media, or a combination thereof. In some embodiments, the media is supplemented with, for example, fetal bovine serum. In some embodiments, the media is supplemented with about 10% fetal bovine serum. In some embodiments, the chamber is flushed with RPMI medium+10% fetal bovine serum (FBS). In some embodiments, the chamber is flushed with a volume of media about equal to the volume of the chamber. In some embodiments, the chamber is flushed with a volume of media about equal to 2, about 3, about 4, about 5, about 6, about 7, about 8, about 9, about 10, about 15, about 20, about 25, about 30, about 35, about 40, about 45, or about 50 times the volume of the chamber.

In some embodiments, an extracellular matrix protein is introduced into the chamber of the device. Suitable extracellular matrix proteins include, but are not limited to, fibronectin, laminin, collagen, MATRIGEL, and combinations thereof. In some embodiments the extracellular matrix protein is fibronectin.

In some embodiments, the extracellular matrix protein is introduced into the chamber of the device after flushing and before filling the compartment of the device with media.

In some embodiments, the extracellular matrix protein is introduced into the chamber of the device in an amount of from about 100 ng/mL to about 100 μg/ml.

In some embodiments, after introducing the extracellular matrix protein in to the chamber of the device, the chamber is incubated at about 37° C. and about 5% CO₂. In some embodiments, the compartment is incubated for about 30 minutes, about 60 minutes, about 90 minutes, about 120 minutes, about 150 minutes, or about 180 minutes.

In some embodiments, the chamber is washed with media after introducing the extracellular matrix protein into the chamber and before filling the chamber with media.

In some embodiments, the chamber is filled with media. Suitable media include any cell-culture media compatible with the target cells, including, but not limited to, serum-free media formulations. In some embodiments, the media is Dulbecco's Modified Eagle Medium (DMEM), Iscove's Modified Dulbecco's Medium (IMDM), RPMI 1640 Media, or a combination thereof. In some embodiments, the media is supplemented with, for example, fetal bovine serum. In some embodiments, the media is supplemented with about 10% fetal bovine serum.

In some embodiments, immune cells are introduced into the chamber of the device. In some embodiments, about 10 to about 3 million immune cells are introduced into the chamber of the device. In some embodiments, about 500,000 to about 3 million, about 1 million to about 3 million, about 1.5 million to about 3 million, about 2 million to about 3 million, about 2.5 million to about 3 million, about 500,000 to about 2.5 million, about 1 million to about 2.5 million, about 1.5 million to about 2.5 million, about 2 million to about 2.5 million, about 500,000 to about 2 million, about 1 million to about 2 million, about 1.5 million to about 2 million, about 500,000 to about 1.5 million, about 1 million to about 1.5 million, about 500,000 to about 1 million, or about 10 to about 500,000 immune cells are introduced into the chamber of the device.

In some embodiments, the microchamber is covered with media after introducing immune cells into the chamber of the device. In this embodiment, the media covering the microchamber is contained within the compartment of the device.

In some embodiments, the compartment is sealed prior to imaging. In some embodiments, the compartment is sealed with a transparent sticky film.

In some embodiments, the chamber is imaged. In some embodiments, the chamber is imaged using time-lapse microscopy. In some embodiments, the chamber is imaged using time-lapse fluorescent microscopy.

In some embodiments, time lapse images are taken about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes.

In some embodiments, images are taken for a total of about 1, 2, 3, 4, 5, 6, 7, 8, 9, 10, 11, 12, 13, 14, 15, 16, 17, 18, 19, 20, 21, 22, 23, or 24 hours.

In some embodiments, the chamber is incubated at about 37° C. and about 5% CO2 during imaging.

In some embodiments, the time-lapse microscopy is time-lapse fluorescent microscopy.

In some embodiments, the images are used to measure the number of target cells on a spot. In some embodiments, the images are used to measure the number of target cells on a spot over time. In some embodiments, the images are used to measure the ratio of immune cells to target cells on a spot. In some embodiments, the images are used to measure trafficking of the immune cells towards the target cells. In some embodiments, the images are used to measure the anti-target cell activity of the immune cells.

Also provided herein are methods of selecting a treatment for a subject having cancer comprising (a) identifying a subject having cancer; (b) generating a plurality of CAR T cells from T cells harvested from the subject; (c) assaying the activity of a subset of the plurality of CART cells by any one of the methods of the disclosure; and (d) selecting a treatment based on the results of said assaying.

In some embodiments, the method comprises identifying a subject having cancer. In some embodiments, the subject is a mammal (e.g., a non-human primate, a human, a mouse, a rodent, or a rabbit). In some embodiments, the subject is human.

In some embodiments, the subject has a cancer selected from acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, Wilms tumor, and combinations thereof.

In some embodiments, a plurality of CAR T cells are generated from T cells harvested from the subject. Methods for producing CAR T cells are known in the art, and described, for example, in Zhang et al., “Engineering CAR-T Cells,” Biomarker Research 5:22 (2017), Vormittag et al., “A Guide to Manufacturing CAR T Cell Therapies,” Curr. Opin. Biotech. 53:164-81 (2018), and Poorebrahim et al., “Production of CAR T-cells by GMP-grade Lentiviral Vectors: Latest Advances and Future Prospects,” Critical Reviews in Clinical Laboratory Sciences 56: 393-419 (2019), each of which is hereby incorporated by reference in its entirety.

In some embodiments, the activity of a subset of the plurality of CAR T cells is assayed by any one of the methods described herein.

In some embodiments, the tumor cells are tumor cells harvested from the subject.

Also provided herein is a method of treating cancer in a subject, wherein the treatment is selected using a method described herein.

### EXAMPLES

The invention is further described in the following examples, which do not limit the scope of the invention described in the claims.

### Example 1—Materials and Methods

### Construction of CAR, T Cell Culture and Transduction

Anti-BCMA and APRIL CAR constructs were synthesized and cloned into a third-generation lentiviral plasmid backbone under the regulation of a human EF-1α promoter. Anti-BCMA CAR bears a CD8 hinge and transmembrane domain, 4-1BB costimulatory domain, and CD3 zeta signaling domain. ARIL CAR bears a 4-1BB transmembrane and costimulatory domains and CD3z signaling domain. Both vectors also contained a second transgene coding for the fluorescent reporter mCherry to facilitate enumeration of transduction efficiency. Human T cells were purified (Stem Cell Technologies, Catalog #15061) from anonymous human healthy donor leukopacs purchased from the MGH blood bank under an IRB-exempt protocol. For primary T-lymphocyte expansions, bulk human T-cells were activated (day 0) using anti-CD3/CD28 Dynabeads (LifeTechnologies), followed by transduction with a lentiviral vector encoding the CAR 24-hours later as described^{[26]}. T cells were cultured in media supplemented with rhIL-2 (20 IU ml⁻¹) beginning on day 0 of culture and were maintained at a constant cell concentration (0.5×10⁶ mL⁻¹) by counting every 2-3 days. T cells were de-beaded at day 10 of culture and functional assays were performed at day 11, after resting overnight.

### Cell Lines and Culture Conditions

Two B-lymphoblast myeloma cell lines, RPMI-8226 and MM.'s, were purchased from American Type Culture Collection (ATCC). Cells were engineered to constitutively express click beetle green (CBG) luciferase/enhanced GFP (eGFP) and then sorted on a FACSAria (BD) to obtain a pure population (CBG-GFP+) (>99%). RPMI-8226 cells were cultured in RPMI media containing 10% fetal bovine serum (FBS), penicillin, and streptomycin. MM.1s cells were cultured in RPMI media supplemented with 20% FBS, penicillin, and streptomycin. MM.1s BCMA knockout cells were generated used CRISPR/Cas9 technology.

### Flow Cytometry

Anti BCMA-PE antibody was used to detect BCMA expression by flow cytometry (clone 19F2, BioLegend). Cells were stained for 30 min in the dark at 4° C. and washed twice in PBS with 2% FBS. DAPI was added to gate in the viable cells before the acquisition. Samples were run on a Fortessa X-20 (BD) and data analyzed with FlowJo (Version 10).

### Microspotting

Poly-L-lysine solution at 0.1% (w/v) (Sigma-Aldrich) and high-molecular-weight cationic ZETAG solution were mixed at a volume ratio of 100:1. The mixture was spiked with FITC-tagged poly-L-lysine (Sigma-Aldrich) for visualization. Using an automatic liquid dispenser (Picospotter, Poly-Pico Technologies LTD), the solution was dispensed into 16 8-by-8 spot arrays on a 3-by-1 inch ultra-clean glass slide (SuperChip Microarray Slides, Thermo-Fisher Scientific). The spots were dried at room temperature for overnight. For optimal adhesion of cells on the spots, the slide should be used between 15-48 h after spotting the material.

### Microfabrication of the Microchamber Membrane

The PDMS membrane was fabricated with the standard soft lithography process. Briefly, a master mold was fabricated in a negative photoresist (SU-8, Microchem) with a height of 300 μm on a 4-inch silicon wafer. PDMS base and curing agent (PDMS, Sylgard 184, Elsworth Adhesives) were mixed thoroughly at a ratio of 10:1 and cast on the wafer. To adapt membrane to the commercialized well frame, we fabricated the membrane with a thickness of 1.5 mm by casting PDMS mixture (13.5 g) on the 4-in wafer. The wafer with the mixture was degassed in a vacuum chamber and then transferred to an oven (80° C.) to cure overnight. After curing, the membrane was diced and peeled from the wafer. Inlets and outlets were created at the four corners of each square chamber with a 1 mm diameter biopsy punch (Harris Uni-Core). The membrane was then treated with oxygen plasma to hydrophilize the PDMS surface.

### Device Assembly and Operation

The spotted slide and the PDMS membrane were manually aligned and assembled with a commercialized 16-well chamber frame (ProPlate, Grace Bio-Labs). Tumor cell sample (204, 25 million mL⁻¹) were loaded in a microchamber with a pipette. After 5 min, cells that were not on the spots were removed by flushing the chamber with RPMI+10% FBS (200 μL). After patterning the tumor cells, fibronectin solution (R&D systems) (20 μL, 10 μg mL⁻¹) was loaded in the microchamber and incubated at 37° C. and 5% CO₂ for 2 h. The microchamber was washed with media (1004) and then covered with media (2004) to prevent evaporation during the experiments. CAR T cell suspension (20 μL) at desired concentrations was loaded into the chamber. Finally, the 16-well chamber frame was sealed with a transparent sticky film and was ready for the time-lapse imaging.

### Time-Lapse Microscopy

Time-lapse fluorescent microscopy was employed to image the migration and antitumor activity of CART cells. Images were taken at 100-300 locations using a 10× or 20× objective with a time interval of 15-30 min using a fully automated Nikon TiE microscope (Micro Device Instruments). The microscope is equipped with a heat chamber which provides 37° C., 5% CO₂ and humidity for long term imaging. Files in .nd2 format were imported into Fiji ImageJ for analysis.

### Image Processing and Data Analysis

Time-lapse images were processed in Fiji ImageJ. The area and roundness of the fluorescent poly-L-lysine spots were measured by setting an automatic threshold to the images, followed by Analyze Particles function. The number of tumor cells on a spot was measured automatically with Trackmate module in ImageJ. We set the ‘estimated cell size’ to 14 μm and 10 μm and ‘intensity threshold’ to 3 and 0.5 for RPMI8226 and MM1s tumor cells, respectively. These parameters were selected based on the cell size and GFP intensity of each cell line. We verified these parameters generate accurate cell counts by comparing the number counted automatically and manually. After confirming the accuracy, we fixed the parameters for all the experiments. The percentage of remaining tumor cells was calculated as the number of cells at a time point over the initial number of cells. The area of tumor cells and CART cells on a spot were measured in ImageJ using a macro. The macro sets automatic thresholds to a stack of fluorescent images in either Triangle or Huang modes and measures the area of GFP (tumor cells) or mCherry (CAR T cells) positive objects in the image stack. The data was imported into Excel. The graphs were plotted using excel, R and GraphPad Prism.

To calculate the ratio of effector to target cells (E:T ratio), we first calculated the area density of CAR-T cells according to the concentration of CAR-T suspension and the dimensions of the chamber. We then calculated the number of CAR-T cells in a 500×500 μm² region. This area is chosen because the 64 tumor islands are arranged into an 8×8 array spaced 500 μm apart. The ratio of CAR-T/tumor cells is calculated the number of CAR-T cells in the 500×500 μm² region over the average number of tumor cells on a spot.

### Example 2—Micropatterned Tumor Arrays

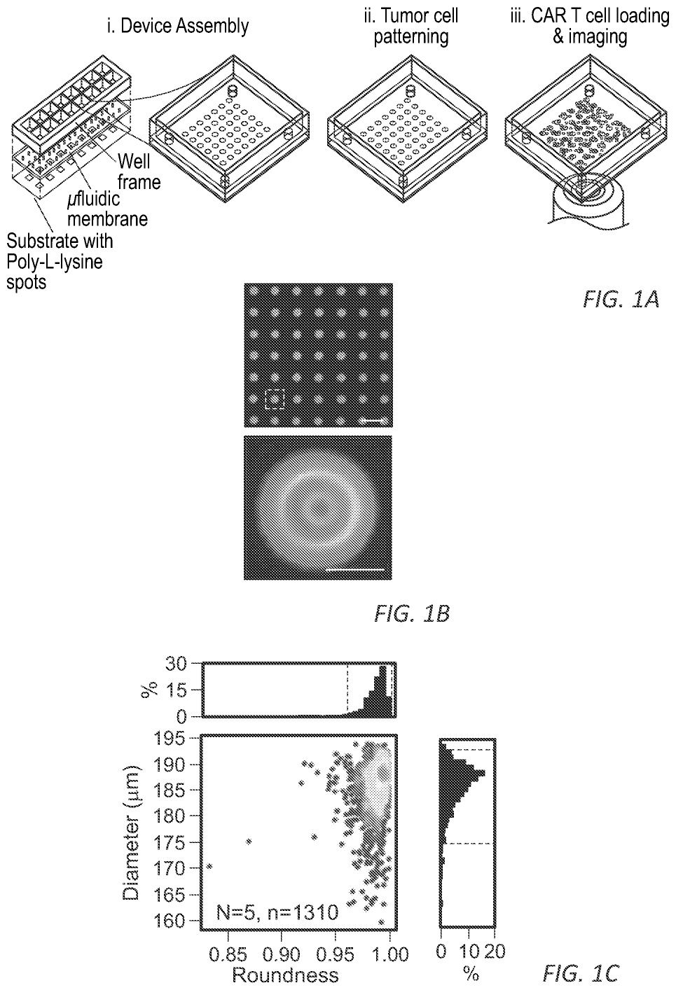

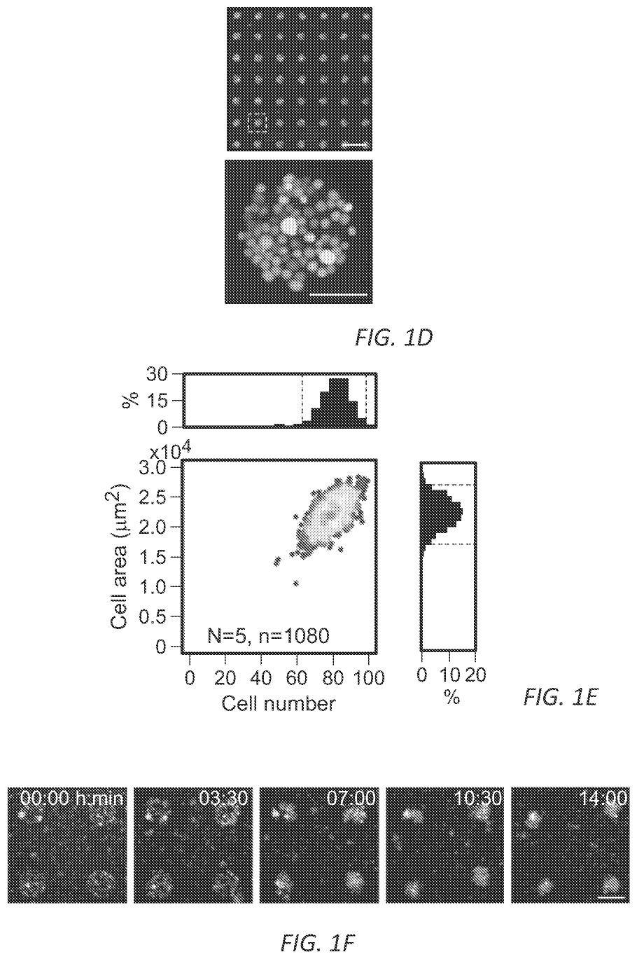

We patterned a large array of microscale tumor-cell islands that are housed in a microfluidic compartment. First, an array of 1024 spots of an adhesion-promoting material that consists of a mixture of Poly-L-Lysine and ZETAG were patterned on 1×3″ glass substrates using an automated liquid dispenser (FIG. 1A, i). The patterned adhesive spots have a uniform diameter of 185.5±5.2 μm and an average roundness index of 0.99±0.01. Then, we assembled on top of the patterned glass substrate, a PDMS membrane containing 16 microfluidic chambers and a plastic frame defining 16-wells (FIG. 1A, i). After assembly, the substrate is divided into 16 individual compartments. Each compartment contains 64 spots spaced 500 μm apart, in an 8×8 array (FIG. 1A, i zoom-in).

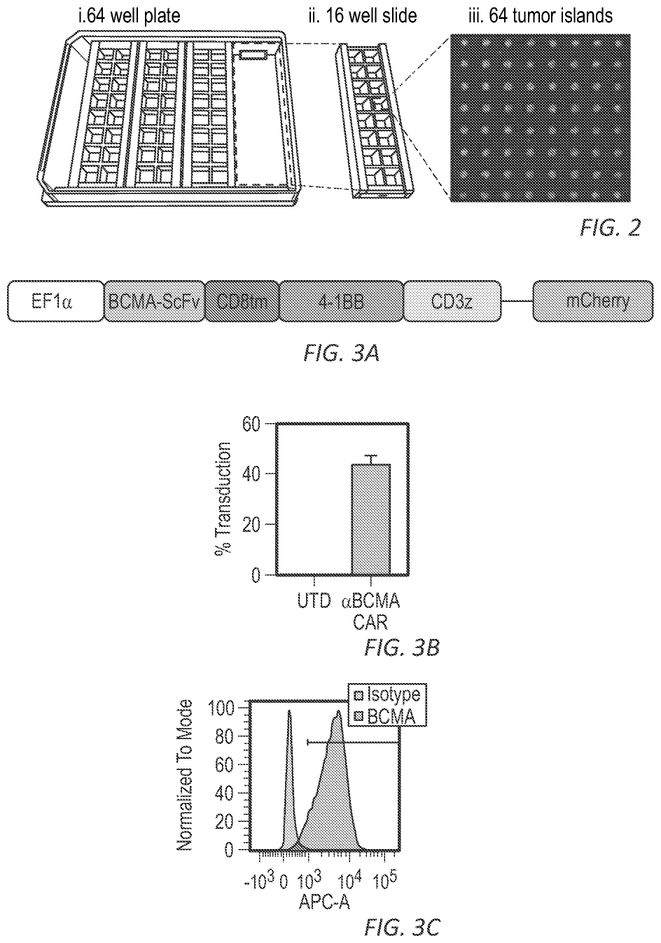

To form the tumor cell islands, we load a suspension of tumor cells inside the microfluidic chambers and allow them to sediment and adhere on the spots. We then remove the non-adhered cells by gentle wash (FIG. 1A, ii). Patterning RPMI 8226 tumor cells yields spot arrays with an average 79±7 cells per spot and an average 2.2±0.2×10⁴ μm² area (FIGS. 1D and 1E). The variation of the cell number and area between spots stems from the heterogeneity of the cell size. Finally, we loaded CAR-T cells inside the microfluidic compartments and allowed to sediment. Immediately, we start monitoring the interactions between CAR-T and tumor cells using time-lapse imaging (FIG. 1A, iii). With the assay, we are capable of quantifying the dynamic interactions between CAR-T and tumor cells (FIG. 1F) on 4096 spots on 4 slides, in 64 different conditions in each experiment (FIG. 2).

### Example 3—Endpoint Evaluation of Overall CAR-T Antitumor Efficacy Using MiTA

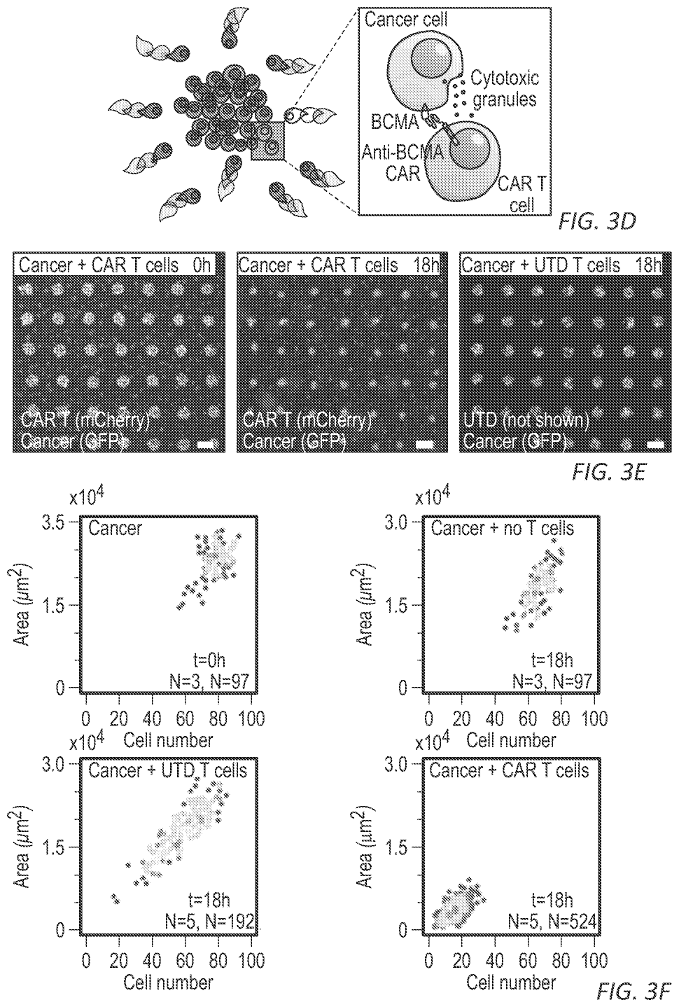

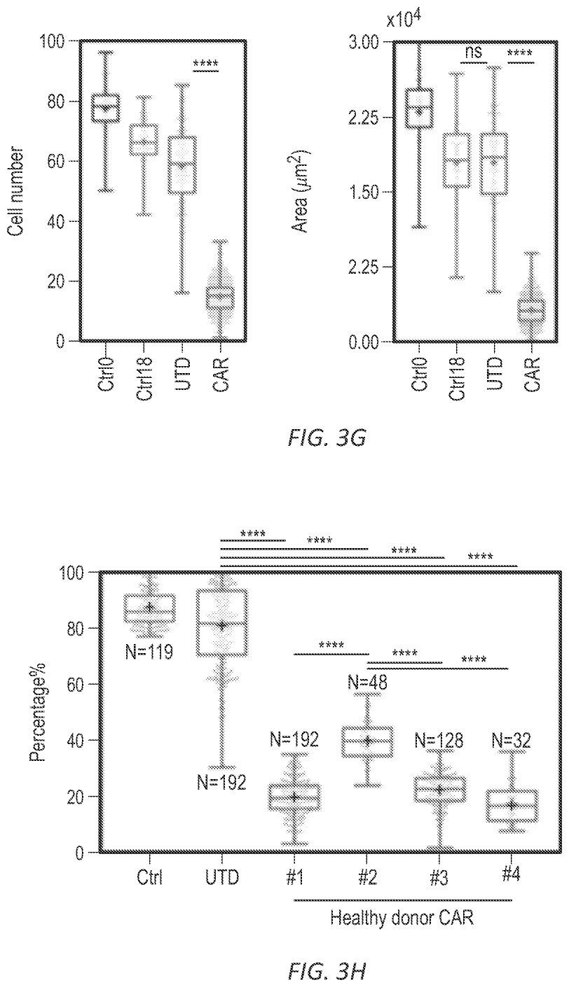

We designed a second-generation anti-BCMA chimeric antigen receptor consisting of a single chain variable fragment (scFv) connected with a CD8 hinge/transmembrane domain to 4-1BB and CD3ζ intracellular domains (FIGS. 3A-3D). In order to facilitate the evaluation of transduction efficiency with the lentiviral construct, we incorporated the mCherry fluorescent reporter gene after a T2A element at the C-terminal of the CAR sequence (FIGS. 3A, 3B). Using flow cytometry, we determined that the efficiency of gene transfer into primary human T cells was 40˜50% (FIG. 3B). We also confirmed high and uniform expression of BCMA antigen by the multiple myeloma (MM) cell line RPMI 8226 by flow cytometry analysis (FIG. 3C). To visualize and distinguish tumor cells from effector CAR-T cells (mCherry positive), we engineered the tumor cells to express the green fluorescent protein (GFP).

A microscopic end-point snapshot of MiTA enables a quantitative evaluation of the overall antitumor efficacy of CAR-T cells. We observed a significant shrinkage of the BCMA+RPMI 8226 tumor spots at 18 h with the presence of anti-BCMA CAR T cells (FIG. 3E). We quantified the efficacy of tumor cell elimination and found that the number of tumor cells decreased ˜5 fold, from an average of 77 cells per spot at t=0 h down to an average of 15 per spot at t=18 h (FIGS. 3F-3H). At the same time, the area of tumor cell spot shrunk ˜7 fold at 18 h (from 2.3×10⁴ at 0 h down to 3.2×10³ μm² per spot at 18 h). In control experiments, untransduced (UTD) T cells were not able to eliminate tumor cells (FIG. 3E). The number of surviving tumor cells were 3.9× and higher than in the presence of CART cells and the tumor area 5.6× higher (FIG. 3G). The much lower percentage of tumor cells survived after 18 h with CART cells than UTD T cells confirms the efficient tumor killing by CART cells (17.1% 39.3% vs. 81.0% live tumor cells at 18 h, CAR-T cells vs. UTD) (FIG. 3H). We also compared CAR-T cells from 4 different healthy donors and found that substantial differences in antitumor activity, ranging from 17.1% (donor 3) to 39.3% (donor 2) (FIG. 3H), despite a similar ˜50% transduction efficiency. Taken together, the end-point snapshots of MiTA unveiled that CAR T cells eliminated the tumor cells more efficiently and consistently than the unspecific killing by UTD T cells and the antitumor efficacy varied among donors.

### Example 4—Dynamic Profiling of the CAR T Antitumor Activity Using MiTA

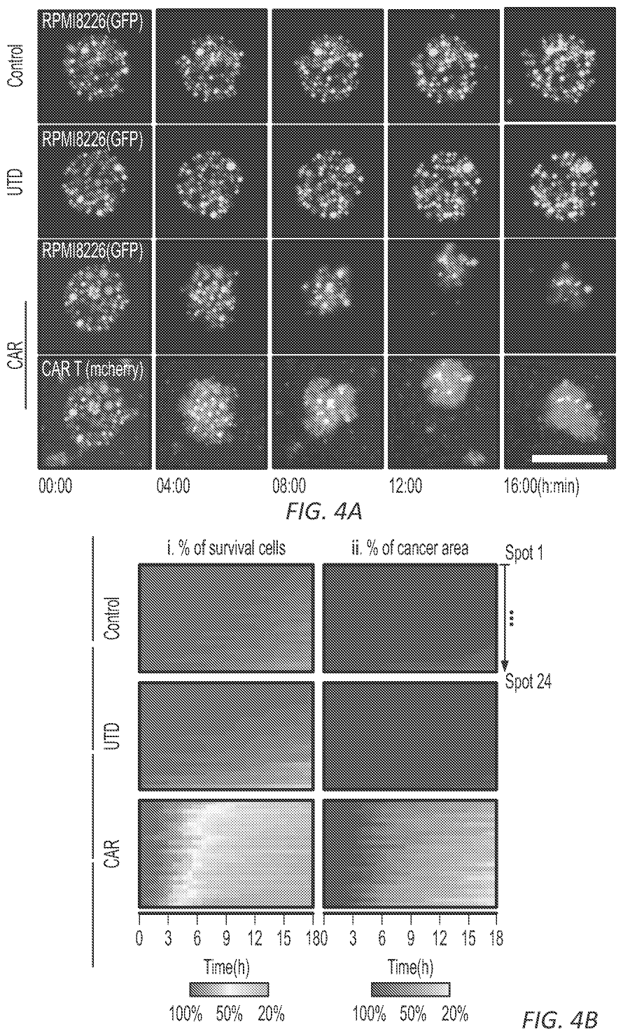

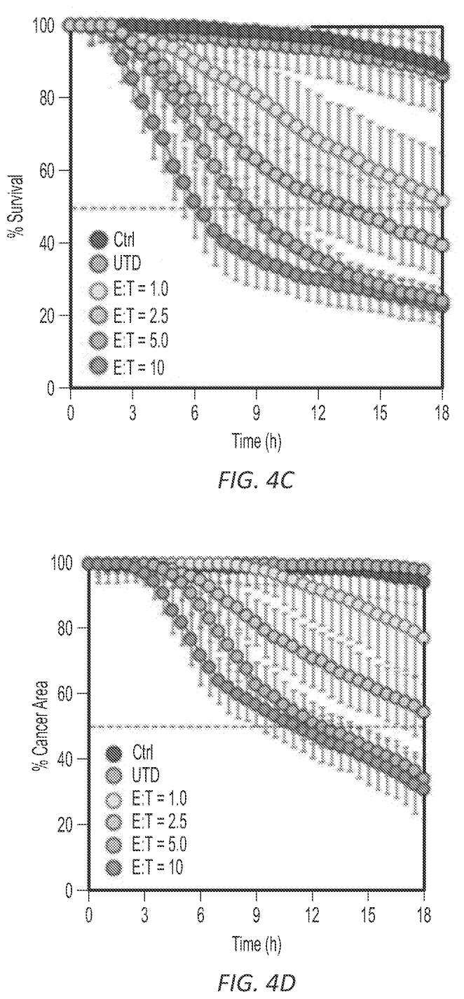

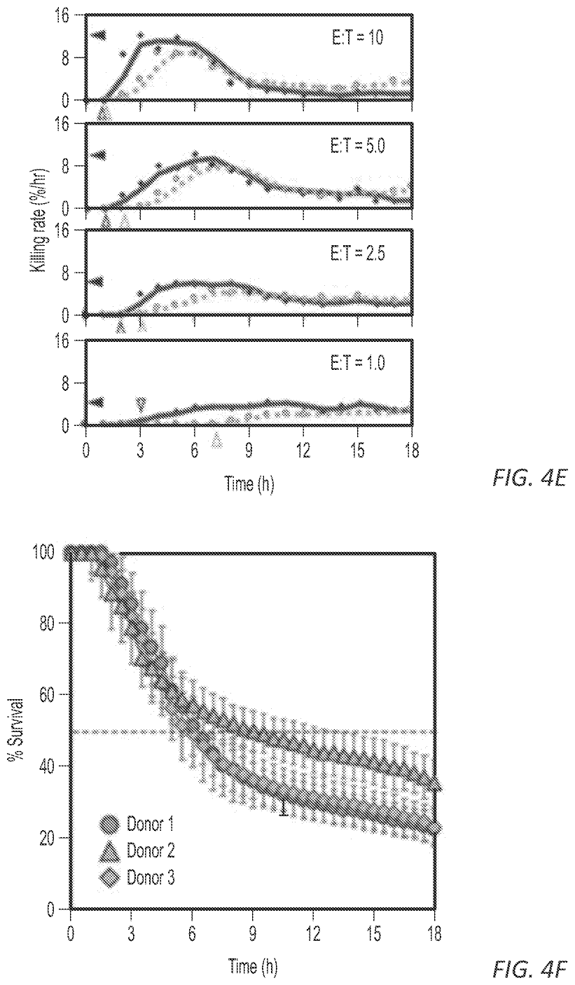

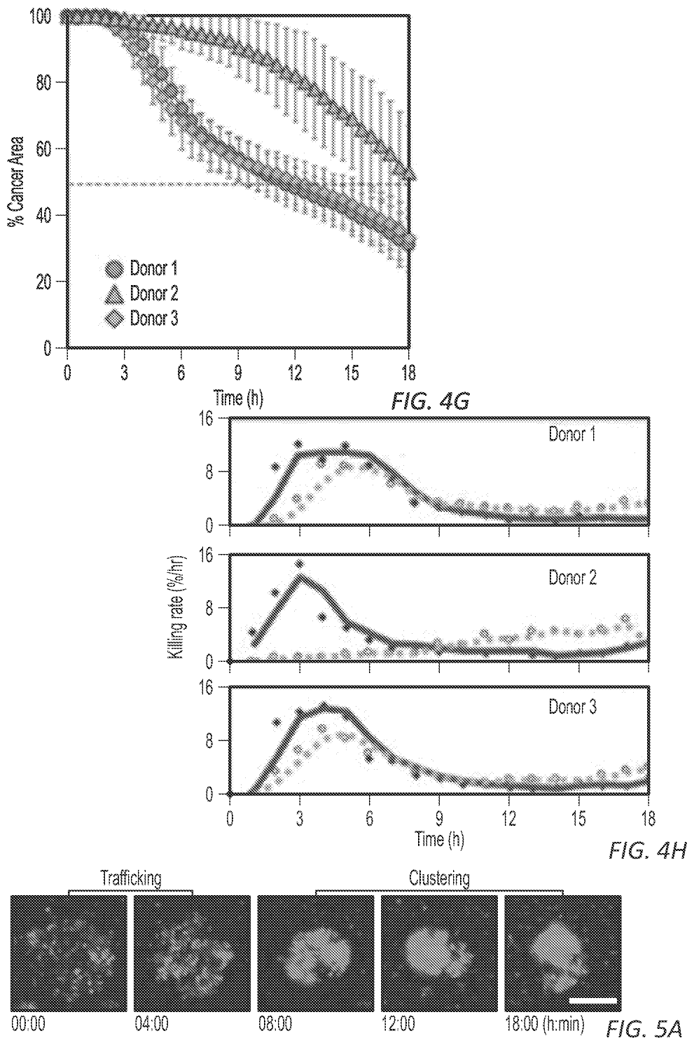

The micropatterned tumor array enables us to visualize and quantify the dynamic process of tumor elimination by CAR T cells (FIGS. 4A-4H). We distinguished two phases during the interaction between CART and tumor cells: an initial phase of CART cell accumulation at the tumor islands followed by a phase of rapid tumor killing. During the initial phase, which lasts 2 h, CAR T cells migrate towards the tumor islands (FIG. 4A, CAR). Most tumor cells stay alive (FIG. 4B, i, CAR) and the initial morphology of the islands is retained (FIG. 4B, ii, CAR). During the second phase, lasting up to 18 h, CAR T cells eliminate tumor cells. During 3-6 h, the killing progresses rapidly, with a peak killing rate at ˜10% cells/h at effector to target cell ratio E:T=10 (FIGS. 4A-4C, CAR; FIG. 4E, solid line, 3-6 h).

The killing of individual tumor cells induces rapid shrinkage of tumor spots (FIGS. 4D and 4E, dashed line). When ˜60% tumor cells were killed, the area of tumor decreased to <40% (FIGS. 4C and 4D, E:T=10). After 6 h, both the killing of individual tumor cells and the shrinkage of the tumor area slow down, with the killing rates gradually decreases to ˜2%/h (FIG. 4E). The tumor cells are often lifted from the spots and carried around by the CAR T cell clusters (FIG. 4A, CAR, 8-18 h). At 18 h, 75% tumor cells are eliminated and the tumor area decreases by 70% (FIGS. 4C and 4D). In contrast, UTD cells failed to kill tumor cells throughout the 18 h. Most tumor cells area alive, and the tumor islands retain their initial morphology (FIGS. 4A-4D, UTD).

The dynamic profile of tumor cell elimination varies with the ratio of effector-to-target cells in two major aspects (FIGS. 4B-4D). Loading more CAR T cells induces an earlier onset of tumor killing and tumor shrinkage. The killing of tumor cells started at 1 h, 1 h, 2 h, and 3 h and the decrease in the tumor area started at 1 h, 2 h, 3 h, and 7 h at E:T=10, 5, 2.5, and 1 respectively (FIG. 4E, hollow arrows). It is worth noting that the time difference between the onsets of tumor killing and island shrinkage also varies with E:T. At E:T=10, the killing and shrinkage occur simultaneously. At E:T=1, the shrinkage of tumor islands follows the killing, 4 h later (FIG. 4E). Loading more CAR T cells accelerates the elimination of tumor cells and the shrinkage of the tumor area. The peak rate of killing decreases from ˜12%/h to ˜4% as E:T decreases from 10 to 1 (FIG. 4E, black arrows) and the time to eliminate 50% tumor cells increases from 6 to 18 h (FIG. 4C, black dashed line). The time to shrink the tumor area by 50% increases from 10 to 12 as the E:T decreases from 10 to 5. For smaller E:T ratios of 2.5 and 1, the tumor area remains >50% at 18 h (FIG. 4D, black dashed line).

### Example 5—CAR T Trafficking Towards Tumor Spots

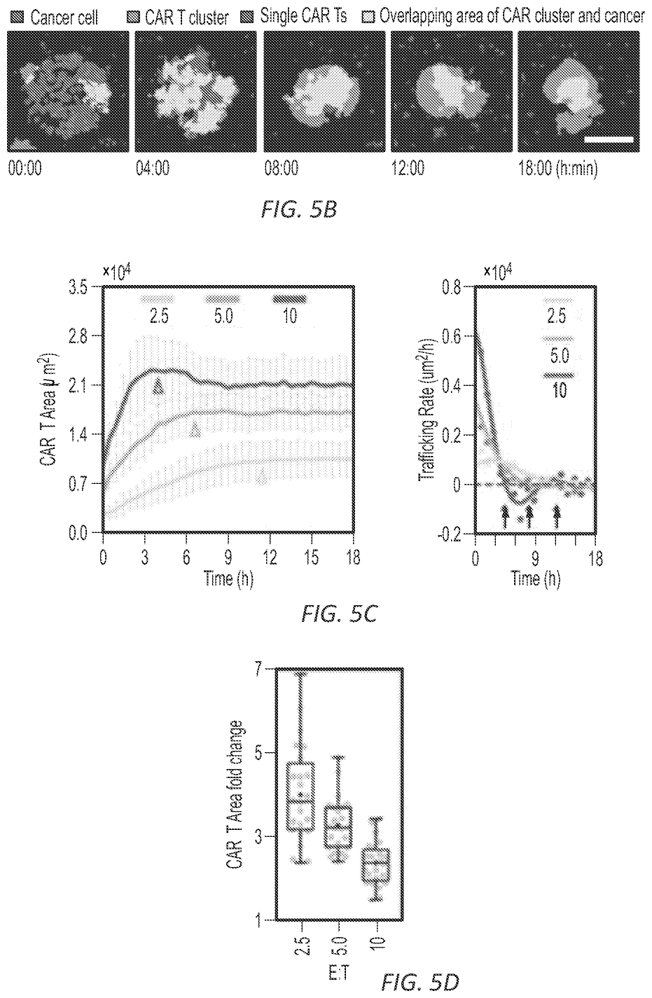

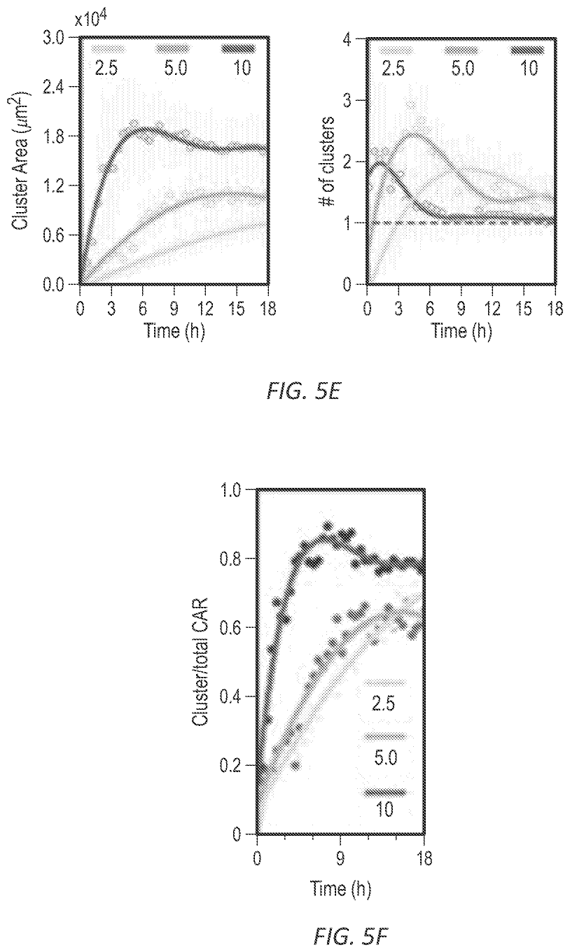

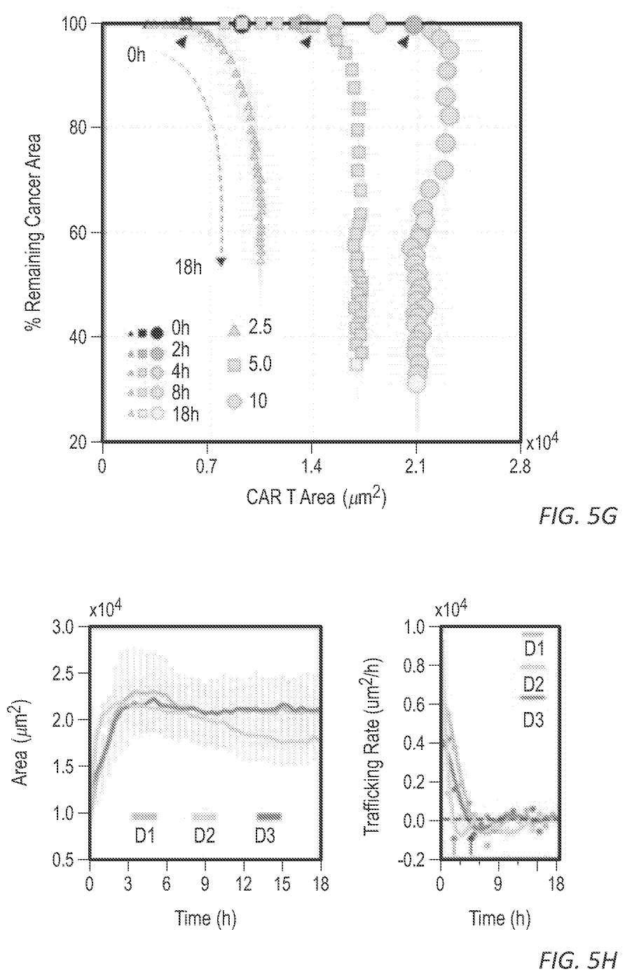

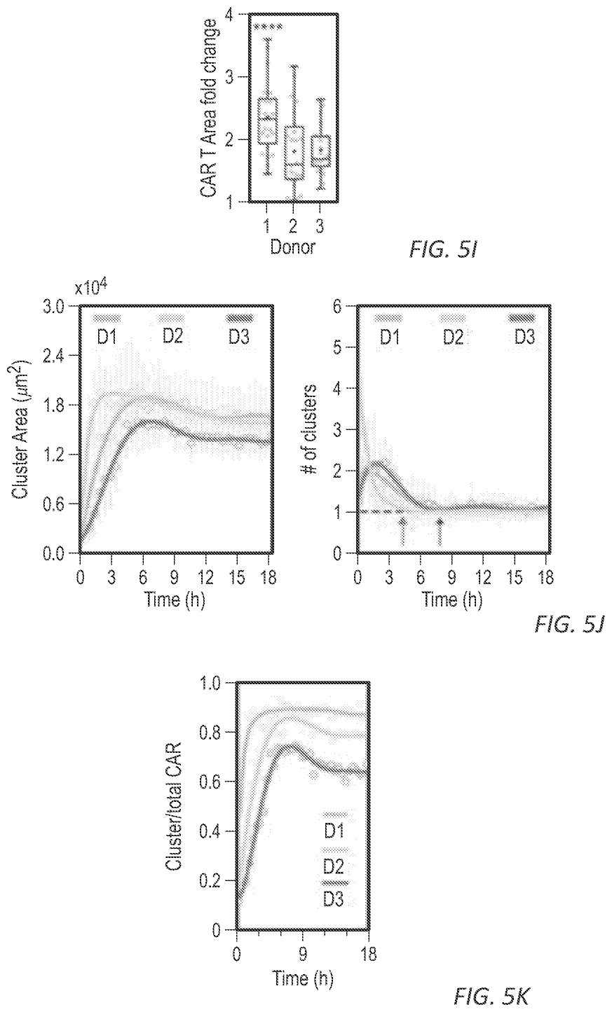

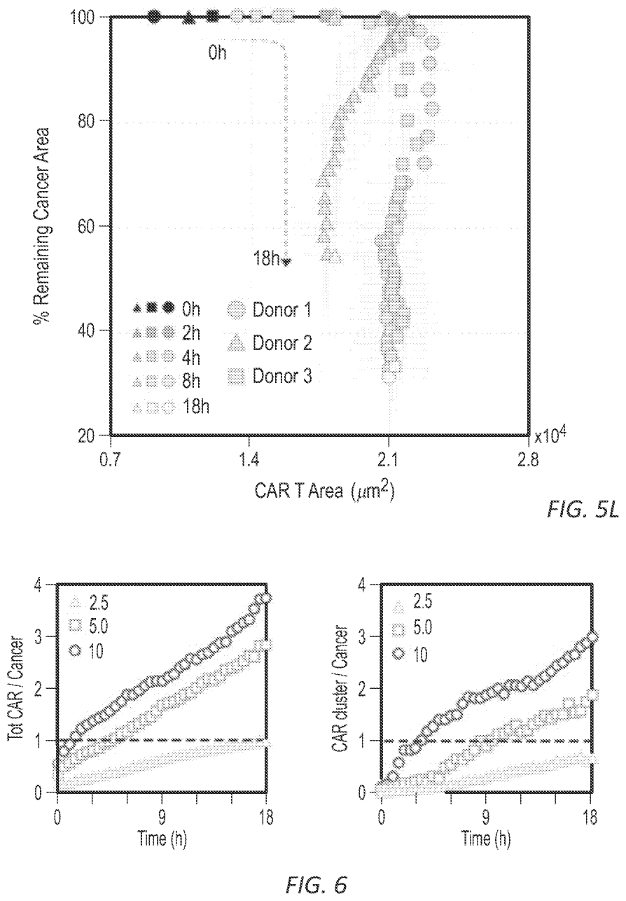

We quantified the trafficking of CAR T cells at various cell densities (FIGS. 5A-5L). We found that a rapid, initial trafficking phase is usually followed by a slow, plateau phase. The duration of the first phase depends on the E:T ratio. At E:T=10 (high CART density), CAR T cells continuously migrated to the spots in the first 4 h with the CAR T cell area increased by 3 fold (FIG. 5C). After 4 h, the trafficking plateaus and the accumulation rate decreases to ˜0 μm²/h (FIG. 5C right panel, black arrow). At lower E:T ratios, the trafficking is slower and the plateaus are delayed (FIG. 5C right panel, 4 h, 6 h and 12 h at E:T=10, 5 and 2.5). The fold change in the CART area is larger at smaller E:T ratios. At 18 h, the area is increased by 4.0×, 3.2× and 2.4× at E:T=2.5, 5 and 10 respectively (FIG. 5D), confirming the robustness of trafficking.

We mapped the dynamic correlation between CAR-T cell trafficking and the killing of tumor cells (FIG. 5G). We found that at higher cell density (E:T=5 and 10), CAR T cells exert efficient killing after the trafficking has plateaued. For example, in the first 2 h, while the CAR T area increases 3×, the area of tumor cells does not change (FIG. 5G E:T=10, circles). From 2-8 h, the CAR T trafficking reaches the plateau and the area of tumor cells shrinks rapidly to 60% and 30% at 8 and 18 hours, respectively.

### Example 6—CAR T Clusters Enhance Tumor-Cell Killing

We observed that CAR-T cells often form large clusters on top of target islands when killing the tumor cells (FIGS. 5A and 5B). During tumor cell killing, CART cells first formed multiple small clusters on top of the tumor island and then merge into one large cell cluster that engulfs the tumor cells within it (FIG. 5B). In the first 6 h, the average cluster area increases from 1600 to 18,000 μm² while the average number of clusters decreasing from 2 to 1, indicating the multiple small clusters merge into one large cluster (FIG. 5E, E:T=10). At 18 h, 80% of the CAR T cells merged into a single cluster on the spot (FIG. 5F, E:T=10) and tumor cells were enveloped within the CAR cluster completely (FIG. 5B). At E:T ratios below 5, the clustering is slower and CAR T cells end up forming multiple smaller clusters (FIGS. 5E and 5F, E:T=2.5 and 5). At 18 h, only 60% of CART cells formed clusters on the tumor island with the rest dispersed around tumor islands (FIG. 5F, E:T=2.5 and 5). The CAR-T cluster to tumor area ratio increases to >2 during 18 h, indicating effective control of the tumor by CAR T (FIG. 6). At low CAR T density (E:T=2.5), the trafficking is slower and overlaps with the killing (FIG. 5G, triangle). The killing is inefficient, and the CAR T clusters do not grow larger than the tumor cell area even at 18 h (FIG. 6).

### Example 7—Heterogeneity in Antitumor Activity of CAR T-Cells from Different Donors

We found distinct dynamic profiles of tumor killing by CAR T cells originating from different healthy donors (FIGS. 4F-4H). Tumor cells were killed equally efficient by CAR T cells from donor 1 and 3. However, tumor cells were killed less efficiently by CAT T cells donor 2 and the shrinkage of tumor islands was slower and delayed (FIGS. 4F-4G, donor 2). Interestingly, tumor cells were killed faster during the first 3 h by the CAR T cells from donor 2, with the highest killing rate of ˜16%/h at 3 h (FIG. 4H, donor 2). However, the killing slowed down after 3 h, with the rate sharply decreasing to ˜4% at 5 h and then ˜2% at 8 h. As a result, only 60% tumor cells were eliminated at 18 h (FIG. 4G). The rate of area shrinkage remains <4% throughout 18 h distinct from the other 2 donors (FIG. 4H, light blue dashed lines).

We found that the CAR T cells from different donors also displayed different trafficking and clustering profiles (FIGS. 5H-5J). CAR T cells from donor 1 exhibited the strongest trafficking with 2.5× increase in the area after 18 h, much higher than donor 2 and 3 (FIG. 5I). In addition, they showed the best ability to cluster around the tumor cells, with ˜90% cells merging in to a single cluster at 18 h (FIG. 5K, blue line). The trafficking of CAR T cells from donor 2 was the fastest but plateaued the earliest among the 3 donors (FIG. 5H, green line). Uniquely, the CAR T area kept decreasing after 3 h, leading to the smallest area at 18 h (FIG. 5H). CAR T cells from donor 2 clustered the fastest and formed a single cluster at 4 h, earlier than the other 2 donors (FIG. 5J, green lines and green arrow). CAR T cells from donor 3 exhibited a similar trafficking profile as donor 1 (FIGS. 5H and 5J, purple and blue lines). However, their ability to form clusters is weaker than the other 2 donors with only 60% of cells clustering together at 18 h (FIG. 5K, purple). Mapping CAR T trafficking and tumor-cell killing together, we found that the killing efficiency may relate to the area of CAR T cells on the spot (FIG. 5L). Despite fast trafficking at the first 2 h (FIG. 5L, red dots), the area of CAR T cells from donor 2 is smaller after 4 h than the other 2 donors. Correspondingly, the killing is less efficient, shown as a higher % of remaining tumor area at 4, 8 and 18 h (FIG. 5L green, blue and yellow triangles vs. circles and squares).

### Example 8—Comparing the Antitumor Activity of Two CAR T-Cell Constructs Using MiTA

We employed MiTA to compare the antitumor activity of anti-BCMA and APRIL-based CAR T cells towards BCMA positive and negative multiple myeloma MM.1s tumor cells. “A proliferation-inducing ligand” (APRIL) is a soluble ligand that can bind BCMA and the transmembrane activator and calcium-modulator and cyclophilin ligand (TACI), two antigens highly expressed on MM cells. We generated an APRIL-based CAR consisting of a truncated APRIL fused to a spacer domain and to the same endodomain used for anti-BCMA CAR construct (anti-BCAR). Our hypothesis is that dual antigen targeting will enhance the tumor cell killing, reduce the incidence of antigen negative escape, and overall therapeutic potential^{[22]}.

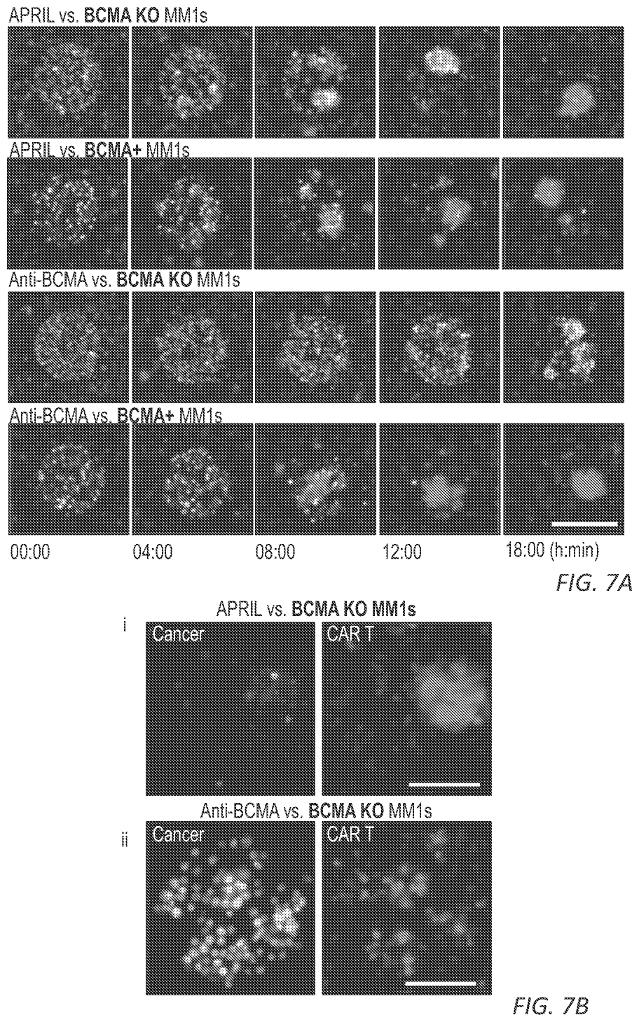

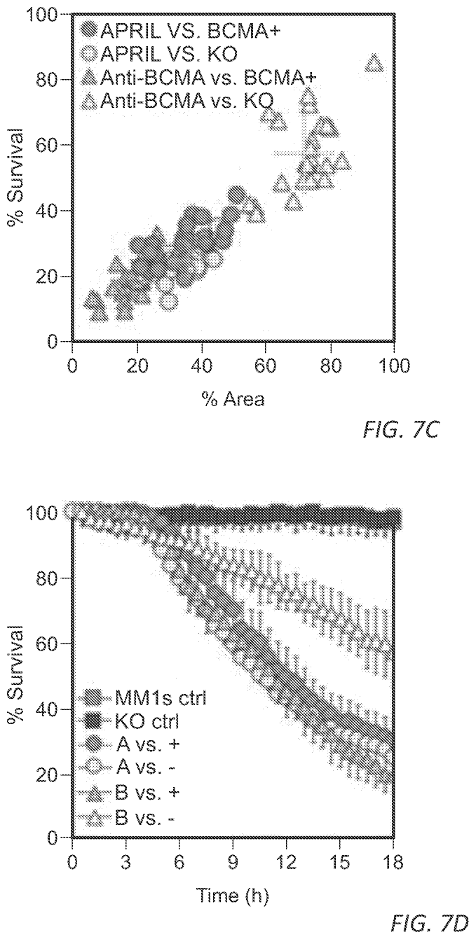

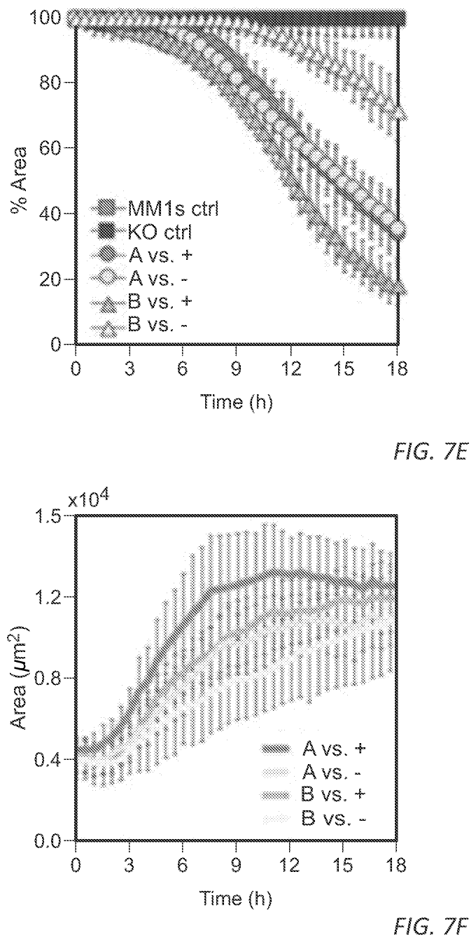

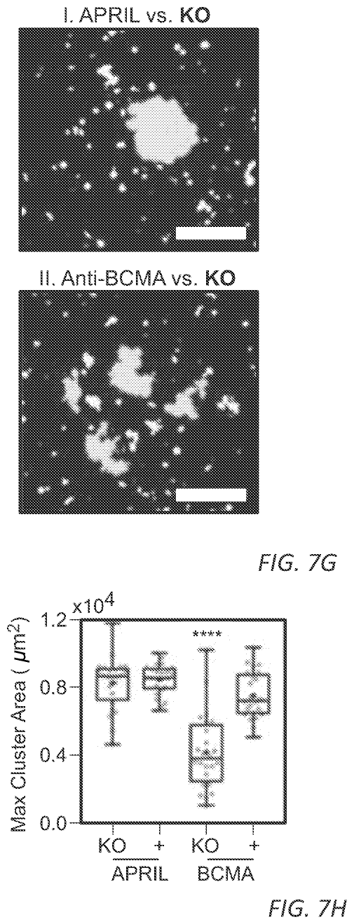

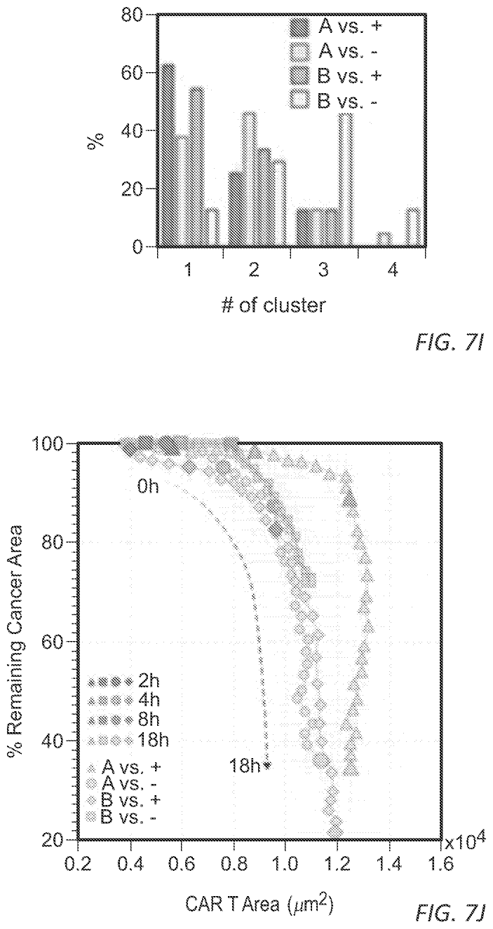

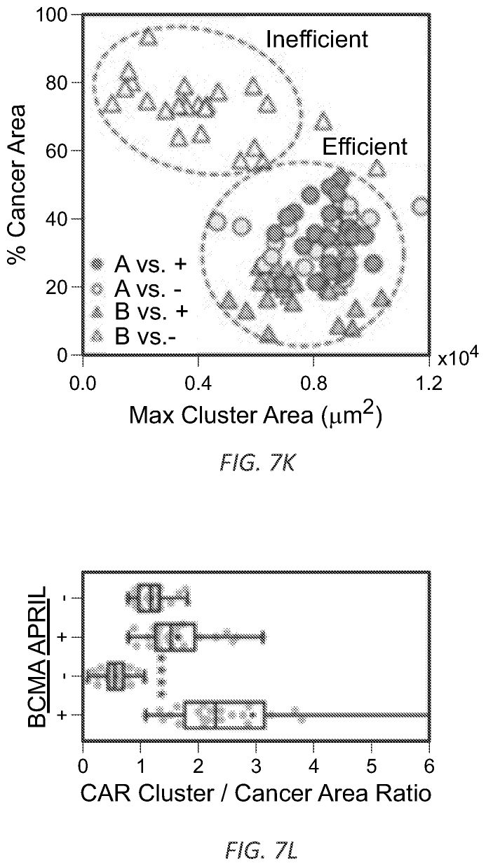

Our assay shows that APRIL-based T cells can efficiently eliminate both BCMA positive and negative MM.1s tumor cells (FIGS. 7A and 7B, I; FIGS. 7C-7E). At 18 h, <30% tumor cells survived the killing and area of the tumor decreased to ˜35% (FIG. 7C). Anti-BCAR T cells eliminate BCMA positive tumor cells more efficiently than APRIL-based CAR T cells (19% surviving tumor cells and 19% remaining area at 18 h), however exhibited significant deficiency in killing BCMA negative tumor cells (FIGS. 7A and 7B, ii; FIGS. 7C-7E). At 18 h, 57% tumor cells had survived the killing and the tumor area had only decreased to 72% (FIG. 7C).

We assessed the dynamic profiles of tumor-cell killing for both CAR T constructs (FIGS. 7D and 7E). APRIL-based CAR T cells exhibited a similar profile for killing BCMA positive and negative MM.1s tumor cells (FIGS. 7D and 7E, dark and light dotted curves) with an interaction phase from 0-3 h and rapid elimination phase after 3 h. Anti-BCAR T cells killed BCMA positive tumor cells more efficiently than APRIL-based CAR T cells, which caused immediate and faster shrinkage of tumor area (FIG. 7E). However, they exhibited deficient killing of BCMA negative tumor cells. The killing started immediately in the absence of the initial interaction stage seen in other conditions (FIG. 7D). The killing rate was slower and the shrinkage of tumor occurred after 9 h, 5 h later than other conditions (FIG. 7E).

We observed different trafficking dynamics for CAR T cells with different constructs (FIG. 7F). The trafficking of APRIL-based CART cells towards BCMA positive tumor spots is the fastest, leading to the largest CAR T area at 9 h among the 4 conditions (FIG. 7F, dark red curve). After 9 h, the trafficking plateaus. The trafficking of APRIL-based CART cells towards BCMA negative tumor cells and anti-BCAR T cells towards BCMA positive tumor cells is slower and plateaus later than 9 hours (FIG. 7F, light red and dark green curves). Finally, the trafficking of anti-BCAR T cells towards BCMA negative tumor cells was the slowest, progressing in a linear fashion from 3 to 18 h (FIG. 7F, light green curve).

Although a similar number of CAR T cells arrive at the spots at 18 hours (FIG. 7F), the clustering of CAR T cells is different for the 4 conditions (FIGS. 7G-7I). When CAR T cells form a single large cluster on the tumor spots, killing is efficient (FIG. 7G, I; FIGS. 7H and &I). When CAR T cells aggregate into multiple, small clusters, killing is inefficient (Anti-BCAR vs. BCMA negative tumor—FIG. 7G, ii; FIGS. 7H and 7I). The largest clusters on the spots in Anti-BCAR vs. BCMA negative tumors are significantly smaller than all other conditions (FIG. 7H). During efficient killing, 37%-63% of the spots have one single large CAR T cluster. In contrast, only 13% of spots have one single cluster in the anti-BCAR CAR T vs. BCMA negative tumor condition. Taken together, our results suggest that APRIL-based CAR can form single, large clusters on both BCMA positive and negative tumor spots. However, anti-BCMA cells failed to form clusters on BCMA negative tumors.

We mapped the correlation between the trafficking of CAR T cells and the corresponding killing which revealed distinct dynamic profiles during efficient and inefficient killing (FIGS. 7J-7I). The results demonstrate distinct dynamics among the 4 conditions and highlighted the deficient killing of anti-BCAR vs. BCMA negative (FIG. 7J, highlighted with a red line) and efficient killing in the other conditions. The clustering of CAR T cells also correlates with the killing efficiency (FIG. 7K). Tumor cells were eliminated efficiently when CAR T cells formed large clusters (FIG. 7K, efficient). In deficient killing situations, CAR T cells only form smaller clusters with a significantly smaller ratio of CAR cluster area to tumor area (FIG. 7I).

In summary, MiTA enables high-content analysis and multi-faceted comparison of the antitumor activity between different CAR T constructs. Our data shows that APRIL-based CAR T cells can effectively migrate to tumor spots, form clusters, and eliminate both BCMA positive and negative tumor cells, while anti-BCAR T cells failed to do so towards BCMA negative tumor cells. This result suggests that APRIL-based CAR T cells could reduce the incidence of antigen-negative escape and thus have stronger therapeutic potential.

### Example 9—Discussion

We developed a micropatterned tumor array (MiTA) that enables high-content and dynamic profiling of the collective antitumor activity of CAR T cells against multiple myeloma tumor cells^{[23]}. Spatially patterning tumor cells into islands induces strong CAR-T trafficking towards tumor targets of similar size and area and allows for simultaneous characterizations of the recruitment of effector cells and elimination of target cells. The microfluidic compartments minimize the mechanical perturbation acting on loosely adherent T cells and prevents artificial cell interactions induced by cell drifting. The integration of the microfluidic compartments in a multi-well plate format facilitates multiplexed and high-throughput screening of CAR-T cells which could expedite the testing of different tumor cell lines, CAR T constructs and drug candidates.

Compared to widely-used biochemical assays that only provide end-point results, MiTA provides comprehensive information regarding CAR T trafficking and subsequent tumor killing. Compared to conventional cell-based assays that probe the interactions of effector and target cells that are randomly distributed on a surface, MiTA enables monitoring of collective interactions of CAR T cells with spatially patterned tumor cell group, which revealed the potential impacts of CAR T cell recruitment and clustering on tumor cluster elimination. Compared to microfluidic and organ-on-a-chip models, MiTA is more straightforward to setup and enables simultaneous characterizations of antitumor activities on a large number of structurally similar tumor islands, which may promote the robustness of screening. Table 1 shows a comparison of features of MiTA (this tool) and other approaches for pre-clinical screening of cancer immunotherapy.

TABLE 1
A table comparing the features of 4 in vitro approaches
for pre-clinical screening of cancer immunotherapy.

| | | | Real-time | |
|---|---|---|---|---|
| | | | monitoring + | Microfluidics/ |
| Features | This tool | Biochemical | cell culture plate | organ-on-a-chip |
| End-point killing | Yes | Yes | Yes | Yes |
| Dynamic killing | Yes | No | Yes | Yes |
| Trafficking of | Yes | No | No | Yes |
| effector cells | | | | |
| Ease of use | Yes | Yes | Yes | No |
| Ease of setup | Yes | Yes | Yes | No |
| Robustness | Yes | Yes | Yes | No |
| Physiological | No | No | No | Yes |
| relevance | | | | |
| Throughput | High | High | High | Low |
| Preparation-to- | <1 day | <1 day | <1 day | >3 days |
| answer time | | | | |
| High-content | Yes | no | no | Yes |

The micropatterned tumor array exhibits high-content information on the dynamic interaction between CAR T cells and tumor islands. The dimensionality of information can be expanded further to decipher this process with greater details. In addition to the area and number of cells and clusters, one could characterize shape factors such as aspect ratio, circularity etc. as well as the correlation between the tumor and CAR T cluster shapes. Ultimately, multiple-dimensional data may be introduced into a machine learning algorithm for better stratifying the efficiency of CAR T cells against tumor cells.

Studying the interaction between different CAR T cells and tumor cell types revealed a signature profile for the antigen-specific killing of tumor cells. Efficient killing driven by antigen-specific binding is characterized by an initial, slow accumulation phase and a subsequent rapid killing phase. In the initial phase, CAR T cells migrated from surrounding to the tumor-cell island but exerted a limited cytolytic effect on the tumor cells. Later, the CAR T cells on the island merged into large clusters and exerted a strong cytolytic effect. In contrast, with inefficient antigen-specific binding (anti-BCAR vs. BCMA negative MM.1s), the interaction lacks the initial phase and the killing is overall slower and less efficient.

The CAR T cell trafficking and clustering around tumor-cell islands highlights the complex interactions involved in efficient killing of tumor cells. Our results confirm that trafficking is a robust phenomenon that is independent of CAR T density. Moreover, trafficking boosts the local ratio of effector to target cells on the niche, facilitating the killing of tumor cells. This finding echoes a recent in vivo observation in a mouse model of B cell lymphoma which showed that the density of CAR T cells increased by 10 fold in 3 days in the bone marrow and the tumor clearance was correlated with the CAR T density^{[24]}. Together, our in vitro data and the in vivo model confirm the importance of CAR T trafficking in promoting tumor cell killing.

When exerting the cytolytic effect on a tumor island, CAR T cells merge into clusters around tumor cells and collectively shrink the tumor island. We found that the size and morphology of the CAR T cell clusters are correlated with the efficiency of clearance of the tumor cells on the island. The formation of a single large CAR T cluster on the island is always associated with better tumor clearance. The formation of multiple smaller clusters, either due to lower CART density or the absence of tumor antigen is related to deficient tumor clearance. These findings imply that efficient clustering of CAR T cells may play an important role in clearing tumor cell clusters. CAR T cell clustering has been recently reported in a mouse model of B cell lymphoma. CAR T cells formed large cell clusters around malignant B cells in the blood circulation 15 min after injection^{[24]}. Whether the cluster formation in vivo promotes tumor cell killing or follows the same dynamics observed in vitro remains to be investigated.

Our data show that APRIL-based CAR-T cells efficiently killed both BCMA positive and negative MM.1s, while anti-BCMA CAR-T cells failed to kill BCMA negative MM.1s. These in vitro data match the results from in vivo experiments which demonstrated that anti-BCMA CAR T cells are unable to clear MM1.s BCMA KO cells engrafted in NSG mice (manuscript under review). These results suggest that our platform could support the validation of CAR T cell efficacy. The trafficking and clustering of April-CART cells occurred in a similar fashion towards both tumor cells, while anti-BCMA CAR-T cells showed a deficient ability to form clusters on BCMA negative MM.1s. These suggest the potential of April-CAR T cells to reduce the incidence of antigen-negative escape, without compromising other key cell functions.

Although it permits multi-faceted dynamic characterizations of CAR T cells with ease of use and high throughput, MiTA is not without limitations. For example, the recruitment of CAR T cells and their interactions with tumor cells happen on a 2D surface which may differ from those in a physiologically relevant 3D microenvironment. This limitation can be overcome by incorporating more features in MiTA. For example, dispensing CAR T cells embedded in hydrogel on top of tumor island array could realize the monitoring of CAR T cell recruitment and cytolytic activities in 3D. Co-patterning tumor cells with other cellular components such as bone marrow stromal cells could provide a more sophisticated in-vivo like microenvironment. Implementing more features in MiTA will shift it towards a more physiologically relevant model but complicate the preparation and the operation of the system at the same time. The versatility of MiTA platform allows possible system modification to adapt to the requirements of different studies and screenings.

The dynamic profiles of CAR T cell trafficking, clustering and tumor elimination vary among healthy donors. The differences may stem from the intrinsic variations in T cell populations among donors or variations induced during CAR T cell manufacturing. The functions of CAR T cells among patients are likely to be poorer and vary even more. Deciphering the link between the variability and the corresponding clinical outcome will facilitate the production of more effective CART cells and could ultimately serve as a biomarker of response or a measure of T cell “fitness”^{[25]}. Ultimately, mapping the dynamic information from in vitro assays, multi-omics data of patients and the clinical outcome could create a landscape that aids the development of more efficient and personalized CAR-T cell therapies.

### REFERENCES

[1] R. J. Brentjens, M. L. Davila, I. Riviere, J. Park, X. Wang, L. G. Cowell, S. Bartido, J. Stefanski, C. Taylor, M. Olszewska, O. Borquez-Ojeda, J. Qu, T. Wasielewska, Q. He, Y. Bernal, I. V. Rijo, C. Hedvat, R. Kobos, K. Curran, P. Steinherz, J. Jurcic, T. Rosenblat, P. Maslak, M. Frattini, M. Sadelain, Sci. Transl. Med. 2013, 5, 177ra38.
[2] R. A. Gardner, O. Finney, C. Annesley, H. Brakke, C. Summers, K. Leger, M. Bleakley, C. Brown, S. Mgebroff, K. S. Kelly-Spratt, V. Hoglund, C. Lindgren, A. P. Oron, D. Li, S. R. Riddell, J. R. Park, M. C. Jensen, Blood 2017, 129, 3322.
[3] J. N. Kochenderfer, S. A. Rosenberg, Nat. Rev. Clin. Oncol. 2013, 10, 267.
[4] S. L. Maude, N. Frey, P. A. Shaw, R. Aplenc, D. M. Barrett, N. J. Bunin, A. Chew, V. E. Gonzalez, Z. Zheng, S. F. Lacey, Y. D. Mahnke, J. J. Melenhorst, S. R. Rheingold, A. Shen, D. T. Teachey, B. L. Levine, C. H. June, D. L. Porter, S. A. Grupp, N. Engl. J. Med 0.2014, 371, 1507.
[5] S. S. Neelapu, F. L. Locke, N. L. Bartlett, L. J. Lekakis, D. B. Miklos, C. A. Jacobson, I. Braunschweig, O. O. Oluwole, T. Siddiqi, Y. Lin, J. M. Timmerman, P. J. Stiff, J. W. Friedberg, I. W. Flinn, A. Goy, B. T. Hill, M. R. Smith, A. Deol, U. Farooq, P. McSweeney, J. Munoz, I. Avivi, J. E. Castro, J. R. Westin, J. C. Chavez, A. Ghobadi, K. V. Komanduri, R. Levy, E. D. Jacobsen, T. E. Witzig, P. Reagan, A. Bot, J. Rossi, L. Navale, Y. Jiang, J. Aycock, M. Elias, D. Chang, J. Wiezorek, W. Y. Go, N Engl. J. Med. 2017, 377, 2531.
[6] J. H. Park, I. Riviere, M. Gonen, X. Wang, B. Senechal, K. J. Curran, C. Sauter, Y. Wang, B. Santomasso, E. Mead, M. Roshal, P. Maslak, M. Davila, R. J. Brentjens, M. Sadelain, N Engl. J. Med. 2018, 378, 449.
[7] S. J. Schuster, J. Svoboda, E. A. Chong, S. D. Nasta, A. R. Mato, O. Anak, J. L. Brogdon, I. Pruteanu-Malinici, V. Bhoj, D. Landsburg, M. Wasik, B. L. Levine, S. F. Lacey, J. J. Melenhorst, D. L. Porter, C. H. June, N. Engl. J. Med. 2017, 377, 2545.
[8] Y. Mi, C. T. t. Hagan, B. G. Vincent, A. Z. Wang, Adv. Sci. (Weinh) 2019, 6, 1801847.
[9] K. T. Brunner, J. Mauel, J. C. Cerottini, B. Chapuis, Immunology 1968, 14, 181.
[10] T. Decker, M. L. Lohmann-Matthes, J. Immunol. Methods 1988, 115, 61.
[11] M. J. Corey, R. J. Kinders, L. G. Brown, R. L. Vessella, J. Immunol. Methods 1997, 207, 43.
[12] T. Kamiya, D. Wong, Y. T. Png, D. Campana, Blood Adv. 2018, 2, 517.
[13] F. Cerignoli, Y. A. Abassi, B. J. Lamarche, G. Guenther, D. Santa Ana, D. Guimet, W. Zhang, J. Zhang, B. Xi, PLoS One 2018, 13, e0193498.
[14] N. Moore, D. Doty, M. Zielstorff, I. Kariv, L. Y. Moy, A. Gimbel, J. R. Chevillet, N. Lowly, J. Santos, V. Mott, L. Kratchman, T. Lau, G. Addona, H. Chen, J. T. Borenstein, Lab Chip 2018, 18, 1844.
[15] E. Biselli, E. Agliari, A. Barra, F. R. Bertani, A. Gerardino, A. De Ninno, A. Mencattini, D. Di Giuseppe, F. Maffei, G. Schiavoni, V. Lucarini, E. Vacchelli, G. Kroemer, C. Di Natale, E. Martinelli, L. Businaro, Sci. Rep. 2017, 7, 12737.
[16] Y. Ando, E. L. Siegler, H. P. Ta, G. E. Cinay, H. Zhou, K. A. Gorrell, H. Au, B. M. Jarvis, P. Wang, K. Shen, Adv. Healthcare Mater. 2019, 8, e1900001.
[17] R. W. Jenkins, A. R. Aref, P. H. Lizotte, E. Ivanova, S. Stinson, C. W. Zhou, M. Bowden, J. Deng, H. Liu, D. Miao, M. X. He, W. Walker, G. Zhang, T. Tian, C. Cheng, Z. Wei, S. Palakurthi, M. Bittinger, H. Vitzthum, J. W. Kim, A. Merlino, M. Quinn, C. Venkataramani, J. A. Kaplan, A. Portell, P. C. Gokhale, B. Phillips, A. Smart, A. Rotem, R. E. Jones, L. Keogh, M. Anguiano, L. Stapleton, Z. Jia, M. Barzily-Rokni, I. Canadas, T. C. Thai, M. R. Hammond, R. Vlahos, E. S. Wang, H. Zhang, S. Li, G. J. Hanna, W. Huang, M. P. Hoang, A. Piris, J. P. Eliane, A. O. Stemmer-Rachamimov, L. Cameron, M. J. Su, P. Shah, B. Izar, M. Thakuria, N. R. LeBoeuf, G. Rabinowits, V. Gunda, S. Parangi, J. M. Cleary, B. C. Miller, S. Kitajima, R. Thummalapalli, B. Miao, T. U. Barbie, V. Sivathanu, J. Wong, W. G. Richards, R. Bueno, C. H. Yoon, J. Miret, M. Herlyn, L. A. Garraway, E. M. Van Allen, G. J. Freeman, P. T. Kirschmeier, J. H. Lorch, P. A. Ott, F. S. Hodi, K. T. Flaherty, R. D. Kamm, G. M. Boland, K. K. Wong, D. Dornan, C. P. Paweletz, D. A. Barbie, Cancer Discov. 2018, 8, 196.
[18] A. Pavesi, A. T. Tan, S. Koh, A. Chia, M. Colombo, E. Antonecchia, C. Miccolis, E. Ceccarello, G. Adriani, M. T. Raimondi, R. D. Kamm, A. Bertoletti, J.C.I. Insight 2017, 2.
[19] A. Sontheimer-Phelps, B. A. Hassell, D. E. Ingber, Nat. Rev. Cancer 2019, 19, 65.
[20] Y. Choi, E. Hyun, J. Seo, C. Blundell, H. C. Kim, E. Lee, S. H. Lee, A. Moon, W. K. Moon, D. Huh, Lab Chip 2015, 15, 3350.
[21] S. N. Bhatia, U. J. Balis, M. L. Yarmush, M. Toner, FASEB J. 1999, 13, 1883.
[22] L. Lee, B. Draper, N. Chaplin, B. Philip, M. Chin, D. Galas-Filipowicz, S. Onuoha, S. Thomas, V. Baldan, R. Bughda, P. Maciocia, E. Kokalaki, M. P. Neves, D. Patel, M. Rodriguez-Justo, J. Francis, K. Yong, M. Pule, Blood 2018, 131, 746.
[23] S. Stifter, E. Babarovic, T. Valkovic, I. Seili-Bekafigo, C. Stemberger, A. Nacinovic, K. Lucin, N. Jonjic, Diagn. Pathol. 2010, 5, 30.
[24] M. Cazaux, C. L. Grandjean, F. Lemaitre, Z. Garcia, R. J. Beck, I. Milo, J. Postat, J. B. Beltman, E. J. Cheadle, P. Bousso, J. Exp. Med. 2019, 216(5):1038-1049
[25] J. Rossi, P. Paczkowski, Y. W. Shen, K. Morse, B. Flynn, A. Kaiser, C. Ng, K. Gallatin, T. Cain, R. Fan, S. Mackay, J. R. Heath, S. A. Rosenberg, J. N. Kochenderfer, J. Zhou, A. Bot, Blood 2018, 132, 804.
[26] I. Scarfo, M. Ormhoj, M. J. Frigault, A. P. Castano, S. Lorrey, A. A. Bouffard, A. van Scoyk, S. J. Rodig, A. J. Shay, J. C. Aster, F. I. Preffer, D. M. Weinstock, M. V. Maus, Blood 2018, 132, 1495.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A cell assay device comprising
a biocompatible substrate having an upper surface supporting a plurality of arrays of spots comprising an adhesion-promoting material;
a biocompatible membrane having top and bottom surfaces and positioned adjacent to the upper surface of the substrate and defining a plurality of chambers within the membrane between the top surface and the bottom surface of the membrane, wherein the membrane comprises at least two openings in the top surface of the membrane into each chamber to provide access to the chambers; and wherein each chamber in the membrane is aligned with a respective one of the arrays of spots on the substrate; and
a frame positioned on the top surface of the membrane, sandwiching the membrane between the frame and the substrate, wherein the frame defines a series of open-ended compartments, one compartment for each of the chambers within the membrane, wherein the compartments are in fluid communication with the chambers via the openings in the membrane.

2. The device of claim 1, wherein the substrate comprises glass, plastic, or silicon.

3. The device of claim 1, wherein the spots are circular, oval, or toroidal in shape, and wherein the adhesion-promoting material comprises one or more of poly-L-lysine polyacrylamide, a cationic polymer, and a cell adhesion molecule.

4. The device of claim 1, wherein the substrate is substantially planar.

5. The device of claim 1, wherein the plurality of chambers are formed into the bottom surface of the membrane.

6. The device of claim 1, wherein the membrane comprises a polymer.

7. The device of claim 6, wherein the polymer comprises one or more of a polydimethylsiloxane, a plastic, or a hydrogel.

8. The device of claim 1, wherein the membrane is permeable.

9. The device of claim 1, wherein the membrane is impermeable.

10. The device of claim 1, wherein the membrane is semi-permeable.

11. The device of claim 1, wherein the membrane is permeable for molecules and particles having a diameter of from about 1 nm to about 100 μm.

12. The device of claim 1, wherein the membrane has a thickness of about 1.5 mm.

13. The device of claim 1, wherein the spots are between about 150 and about 200 μm in diameter.

14. The device of claim 1, wherein the total number of spots is between about 1 and about 3,000.

15. The device of claim 1, wherein the distance between spots on an array is at least about 35 μm.

16. The device of claim 1, wherein the number of spots on each array is between about 4 and about 100.

17. The device of claim 1, wherein the height of the chamber is between about 50 to about 150 μm.

18. The device of claim 1, wherein the width and depth of the chamber are about 6.4 mm.

19. (canceled)

20. A method of assaying the activity of immune cells on target cells, the method comprising:
introducing the target cells into the chamber of the device of claim 1 through one of the at least two membrane opening;
permitting the introduced tumor cells to settle onto the adhesive dots and adhere thereto;
flushing the device to remove non-adherent tumor cells;
introducing an extracellular matrix protein into the chamber of the device;
filling the chamber of the device with media;
introducing the T cells into the chamber of the device; and
imaging the chamber.

21.-36. (canceled)

37. A method of selecting a treatment for a subject having cancer, the method comprising:
(a) identifying a subject having cancer;
(b) generating a plurality of CART cells from T cells harvested from the subject;
(c) assaying the activity of a subset of the plurality of CART cells by the method of claim 20; and
(d) selecting a treatment based on the results of said assaying.

38. (canceled)
